(19) United States
(12) Patent Application Publication
(10) Pub. No.: US 2026/0265250 A1
(43) Pub. Date: Sep. 10, 2026

(54) 3-ALKYLAMINE INDOLE ACTIVATORS OF SEROTONIN RECEPTORS

(71) Applicant: Atai Therapeutics, Inc., New York, NY (US)

(72) Inventors: Tanweer A. KHAN, Berlin (DE); Glenn F. SHORT, Encinitas, CA (US); Robert B. PERNI, Marlborough, MA (US)

(21) Appl. No.: 19/165,703

(22) PCT Filed: Mar. 13, 2024
(86) PCT No.: PCT/US2024/019770

§ 371 Date: Sep. 16, 2025

Related U.S. Application Data
(60) Provisional application No. 63/452,562, filed on Mar. 16, 2023.

(51) Int. Cl. C07D 471/04; A61K 31/4045; A61K 31/416; A61K 31/4184; A61K 31/423; A61K 31/437; C07D 209/16; C07D 231/56; C07D 235/06; C07D 263/58

(52) U.S. Cl. CPC C07D 471/04; A61K 31/4045; A61K 31/416; A61K 31/4184; A61K 31/423; A61K 31/437; C07D 209/16; C07D 231/56; C07D 235/06; C07D 263/58

(57) ABSTRACT

Provided herein are compounds of Formula (I) and Formula (II), or pharmaceutically acceptable salt thereof, wherein R₁, R₂, R₃, R₄, R₅, R₆, R₇, R₈, R₉, R₁₀, A, B, and E are defined herein. Also provided are pharmaceutical compositions comprising a compound of Formula (I) and Formula (II) or pharmaceutically acceptable salt thereof, and methods of using a compound of Formula (I) and Formula (II) or pharmaceutically acceptable salt thereof, e.g., in the treatment of a mental health disease or disorder.

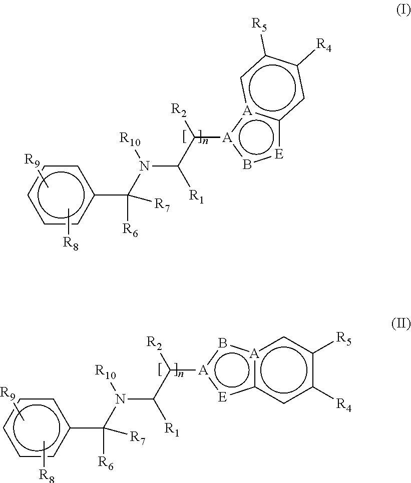

### CROSS-REFERENCE TO RELATED APPLICATIONS

This application claims the benefit of U.S. Provisional Patent Application Ser. No. 63/452,562, filed on Mar. 16, 2023 which is hereby incorporated by reference in its entirety.

### BACKGROUND

Psychedelics can be classified into three main classes: indoleamines, phenylalkylamines, and ergolines. The first class, indolamines includes N,N-dimethyltryptamine (DMT), 5-methoxy-DMT (5-MeO-DMT), psilocy bin and 4-hydroxy-DMT. The second class, phenylalkylamines, includes mescaline, as well as synthetic mescaline analogs such as 2,5-dimethoxy-4-iodoamphetamine (DOI) and 2,5-dimethoxy-4-bromoamphetamine (DOB). The third class are ergolines, such as LSD. The phenylalkylamines are selective agonists of 5-HT2 receptors, including 5-HT2A, 5-HT2B and 5-HT2C receptors. The indoleamines and ergolines act as partial agonists of 5-HT1, 5-HT2, 5-HT6 and 5-HT7 receptors. LSD and other ergolines also act upon D1 and D2 dopamine receptors and adrenergic receptors.

Activation of 5-HT2A receptors located in cortical and subcortical structures of the brain are thought to mediate the subjective, behavioral, and psychological effects of psychedelics in both animals and humans. Serotonergic psychedelics have demonstrated potential for treating a range of mental health diseases or disorders.

There remains a need for compounds that act as agonists of serotonin receptors, such as the 5-HT2A receptor as well as compositions and methods of use thereof.

### SUMMARY OF THE DISCLOSURE

In an aspect the present disclosure provides compounds which act as agonists of serotonin receptors e.g., the 5-HT2A receptor, as well as compositions and methods of use thereof such as, for example, for the treatment of a mental health disease or disorder.

In an aspect, the disclosure provides a compound of Formula (I):

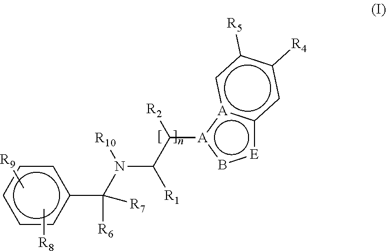

or a pharmaceutically acceptable salt thereof, wherein, R₁, R₂, R₆, R₇, and independently are hydrogen, deuterium, alkyl, deuterated alkyl, heteroalkyl, haloalkyl, cycloalkyl, or heterocyclyl; R₄, R₅, R₈, and R₉ independently are hydrogen, deuterium, alkyl, heteroalkyl, haloalkyl, cycloalkyl, CO₂R₁₁, CONHR₁₁, CO₂N(R₁₁)(R₁₂), CN, or halogen, wherein R₁₁ and R₁₂ independently are hydrogen, deuterium, heteroalkyl, haloalkyl, cycloalkyl, or heterocyclyl; R₁₀ is hydrogen, deuterium, or alkyl; A is C or N; B is CR₁₃, N, or CO, wherein R₁₃ is hydrogen, deuterium, alkyl, heteroalkyl, haloalkyl, cycloalkyl, or heterocyclyl; E is CR₃, NR₃, N, O, or S; wherein R₃ is hydrogen, deuterium, alkyl, deuterated alkyl, heteroalkyl, haloalkyl, cycloalkyl, or heterocyclyl; and n is 0 or 1.

In some embodiments relating to compounds of Formula (I), the compound of Formula (I) is a compound of Formula (I-A):

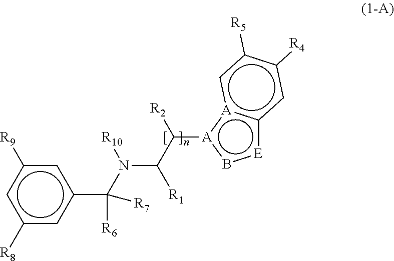

or a pharmaceutically acceptable salt thereof, wherein, R₁, R₂, R₆, R₇, and independently are hydrogen, deuterium, alkyl, deuterated alkyl, heteroalkyl, haloalkyl, cycloalkyl, or heterocyclyl; R₄, R₅, R₈, and R₉ independently are hydrogen, deuterium, alkyl, heteroalkyl, haloalkyl, cycloalkyl, CO₂R₁₁, CONHR₁₁, CO₂N(R₁₁)(R₁₂), CN, or halogen, wherein R₁₁ and R₁₂ independently are hydrogen, deuterium, heteroalkyl, haloalkyl, cycloalkyl, or heterocyclyl; wherein when one A is N, R& and R₉ are not both hydrogen; R₁₀ is hydrogen, deuterium, or alkyl; A is C or N; B is CR₁₃, N, or CO, wherein R₁₃ is hydrogen, deuterium, alkyl, heteroalkyl, haloalkyl, cycloalkyl, or heterocyclyl; E is CR₃, NR₃, N, O, or S; wherein R₃ is hydrogen, deuterium, alkyl, deuterated alkyl, heteroalkyl, haloalkyl, cycloalkyl, or heterocyclyl; and n is 0 or 1.

In some embodiments, compounds of Formula (I) can include compounds according to any of Formulas (I-A), (I-B), (I-C), (I-D), (I-E), and (I-F), as described herein. Thus, in some embodiments relating to compounds of Formula (I), R₁, R₂, R₃, R₆, and R₇ independently are C₁-C₆ alkyl, C₁-C₆ heteroalkyl, C₁-C₆ haloalkyl, C₃-C₇ cycloalkyl, or C₃-C₇ heterocyclyl; R₄, R₅, R₈, and R₉ independently are C₁-C₆ alkyl, C₁-C₆ heteroalkyl, C₁-C₆ haloalkyl, C₃-C₇ cycloalkyl, CO₂R₁₁, CONHR₁₁, or CO₂N(R₁₁)(R₁₂); wherein R₁₁ and R₁₂ independently are C₁-C₆ heteroalkyl, C₁-C₆ haloalkyl, C₃-C₇ cycloalkyl, or C₃-C₇ heterocyclyl; R₁₀ is C₁-C₆ alkyl; B is CR₁₃; wherein R₁₃ is C₁-C₆ alkyl, C₁-C₆ heteroalkyl, C₁-C₆ haloalkyl C₃-C₇, cycloalkyl, or C₃-C₇ heterocyclyl.

In some embodiments relating to compounds of Formula (I), n is 1.

In some embodiments relating to compounds of Formula (I), R₁ is hydrogen.

In some embodiments relating to compounds of Formula (I), R₁ is C₁-C₆ alkyl.

In some embodiments relating to compounds of Formula (I), R₁ is methyl.

In some embodiments relating to compounds of Formula (I), B is CR₁₃.

In some embodiments relating to compounds of Formula (I), R₁₃ is C₁-C₆ alkyl.

In some embodiments relating to compounds of Formula (I), R₁₃ is methyl.

In some embodiments relating to compounds of Formula (I), R₁₃ is hydrogen.

In some embodiments relating to compounds of Formula (I), B is CO. In other embodiments, B is N.

In some embodiments relating to compounds of Formula (I), one A is N. In other embodiments, one A is N and E is N. In other embodiments, one A is N and E is O.

In some embodiments relating to compounds of Formula (I), E is CR; or NR₃, wherein R₃ is hydrogen.

In some embodiments relating to compounds of Formula (I), R₃ is C₁-C₆ alkyl.

In some embodiments relating to compounds of Formula (I), E is CR₃ or NR₃, wherein R₃ is methyl. In other embodiments, R₃ is ethyl.

In some embodiments relating to compounds of Formula (I), R₄ and R₅ are hydrogen.

In some embodiments relating to compounds of Formula (I), R₄ is halogen.

In some embodiments relating to compounds of Formula (I), R₄ is I.

In some embodiments relating to compounds of Formula (I), R₅ is methoxy.

In some embodiments relating to compounds of Formula (I), R₆ and R₇ are hydrogen.

In some embodiments relating to compounds of Formula (I), R₁ is hydrogen.

In some embodiments relating to compounds of Formula (I), R₁ is C₁-C₆ alkyl.

In some embodiments relating to compounds of Formula (I), R₁ is methyl.

In some embodiments relating to compounds of Formula (I), R₈ is C₁-C₆ alkyl.

In some embodiments relating to compounds of Formula (I), R₈ is methyl.

In some embodiments relating to compounds of Formula (I), R₉ is halogen.

In some embodiments relating to compounds of Formula (I), R₉ is Cl.

In some embodiments relating to compounds of Formula (I), R₉ if F.

In some embodiments relating to compounds of Formula (I), R₁₀ is C₁-C₆ alkyl.

In some embodiments relating to compounds of Formula (I), A and A are not simultaneously N.

In another aspect, the disclosure provides a compound of Formula (II):

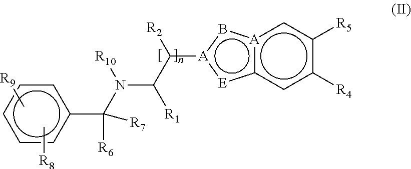

or a pharmaceutically acceptable salt thereof, wherein, R₁, R₂, R₆, R₇, and independently are hydrogen, deuterium, alkyl, deuterated alkyl, heteroalkyl, haloalkyl, cycloalkyl, or heterocyclyl; R₄, R₅, R₈, and R₉ independently are hydrogen, deuterium, alkyl, heteroalkyl, haloalkyl, cycloalkyl, CO₂R₁₁, CONHR₁₁, CO₂N(R₁₁)(R₁₂), CN, or halogen, wherein R₁₁ and R₁₂ independently are hydrogen, deuterium, heteroalkyl, haloalkyl, cycloalkyl, or heterocyclyl; R₁₀ is hydrogen, deuterium, or alkyl; A is C or N; B is CR₁₃, N, or CO, wherein R₁₃ is hydrogen, deuterium, alkyl, heteroalkyl, haloalkyl, cycloalkyl, or heterocyclyl; E is CR₃, NR₃, N, O, or S; wherein R₃ is hydrogen, deuterium, alkyl, deuterated alkyl, heteroalkyl, haloalkyl, cycloalkyl, or heterocyclyl; and n is 0 or 1.

In some embodiments relating to compounds of Formula (II), R₁, R₂, R₃, R₆, and R₇ independently are C₁-C₆ alkyl, C₁-C₆ heteroalkyl, C₁-C₆ haloalkyl, C₃-C₇ cycloalkyl, or C₃-C₇ heterocyclyl; R₄, R₅, R₈, and R₉ independently are C₁-C₆ alkyl, C₁-C₆ heteroalkyl, C₁-C₆ haloalkyl, C₃-C₇ cycloalkyl, CO₂R₁₁, CONHR₁₁, or CO₂N(R₁₁)(R₁₂); wherein R₁₁ and R₁₂ independently are C₁-C₆ heteroalkyl, C₁-C₆ haloalkyl, C₃-C₇ cycloalkyl, or C₃-C₇ heterocyclyl; R₁₀ is C₁-C₆ alkyl; B is CR₁₃; wherein R₁₃ is C₁-C₆ alkyl, C₁-C₆ heteroalkyl, C₁-C₆ haloalkyl C₃-C₇, cycloalkyl, or C₃-C₇ heterocyclyl.

In some embodiments relating to compounds of Formula (II), n is 1.

In some embodiments relating to compounds of Formula (II), R₁ is hydrogen.

In some embodiments relating to compounds of Formula (II), B is N.

In some embodiments relating to compounds of Formula (II), one A is N.

In some embodiments relating to compounds of Formula (II), E is CR₃ or NR₃, wherein R₃ is hydrogen.

In some embodiments relating to compounds of Formula (II), R₄ and R₅ are hydrogen.

In some embodiments relating to compounds of Formula (II), R₆ and R₇ are hydrogen.

In some embodiments relating to compounds of Formula (II), R₈ is C₁-C₆ alkyl.

In some embodiments relating to compounds of Formula (II), R₈ is methyl.

In some embodiments relating to compounds of Formula (II), R₉ is halogen.

In some embodiments relating to compounds of Formula (II), R₉ is Cl.

In some embodiments relating to compounds of Formula (II), R₂ is hydrogen.

In some embodiments relating to compounds of Formula (II), R₁₀ is hydrogen.

In some embodiments relating to compounds of Formula (II), A and A are not simultaneously N.

In another aspect the disclosure provides a pharmaceutical composition, comprising any of the compounds of the above-described aspects and embodiments, and a pharmaceutically acceptable carrier.

In another aspect the disclosure provides a method for treating one or more conditions that are responsive to serotonin receptor activation, comprising administering to a subject in need thereof an effective amount of the compound of any of the above described aspects and embodiments.

In another aspect the disclosure provides a method for treating a neurological disorder, comprising administering to a subject in need thereof an effective amount of a compound of any of the above described aspects and embodiments. In further embodiments, the neurological disorder is major depressive disorder (MDD), treatment resistant depression (TRD), substance use disorder (SUD), an anxiety disorder including acute stress disorder agoraphobia, generalized anxiety disorder, obsessive-compulsive disorder, panic disorder, posttraumatic stress disorder, separation anxiety disorder, social phobia, or specific phobia; an eating disorder including anorexia nervosa or bulimia nervosa; or rumination.

In another aspect the disclosure provides a method for activating a 5-HT receptor comprising administering to a patient a compound of Formula (I) or Formula (II). In some aspects, the 5-HT receptor is a 5-HT2A, 5-HT2B, or 5-HT2C receptor.

Additional aspects and embodiments in accordance with the disclosure will be apparent to one of skill in the art in light of the following description.

### DETAILED DESCRIPTION OF PREFERRED EMBODIMENTS

Before the disclosed methods and materials are described, it is to be understood that the aspects described herein are not limited to specific embodiments, and can vary. It is also to be understood that the terminology used herein is for the purpose of describing particular aspects only and, unless specifically defined herein, is not intended to be limiting.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

The following terms and expressions used herein have the indicated meanings.

Terms used herein may be preceded and/or followed by a single dash, “-”, or a double dash, “═”, to indicate the bond order of the bond between the named substituent and its parent moiety; a single dash indicates a single bond and a double dash indicates a double bond. In the absence of a single or double dash it is understood that a single bond is formed between the substituent and its parent moiety.

The term “about” when used before a numerical designation, e.g., pH, temperature, amount, or concentration, indicates an approximation which may vary by amounts that do not have any significant effect on the resulting structure, stability, activity, or result-effective variable or parameter. In some embodiments the term about can allow for various relative amounts of the particular element or variable such as, for example, of up to (+) or (−) 5% or even up to (+) or (−) 10%.

An “alkyl” group refers to a fully saturated straight or branched chain hydrocarbon containing from 1 to 12 carbon atoms, which is attached to a molecule by a single bond. Alkyl groups can include C₁-C₁₂ alkyl, C₁-C₁₀ alkyl, C₁-C₆ alkyl, C₁-C₈alkyl all of which are inclusive of C₄ alkyls, C₃ alkyls, C₂ alkyls and C₁ alkyl(methyl). Non-limiting examples of alkyl include methyl, ethyl, n-propyl, iso-propyl, n-butyl, sec-butyl, iso-butyl, tert-butyl, n-pentyl, isopentyl, neopentyl, t-amyl, n-hexyl, 3-methylhexyl, 2,2-dimethylpentyl, 2,3-dimethylpentyl, n-heptyl, n-octyl, n-nonyl, n-decyl, n-undecyl, and n-dodecyl. In accordance with some example embodiments an alkyl group can be optionally substituted.

“Alkylene” refers to a saturated, straight or branched bivalent alkyl group. An “alkylene chain” refers to a polymethylene group, i.e., —(CH₂)ₙ—, wherein n is a positive integer which, in certain embodiments, can be from one to six, from one to four, from one to three, from one to two, or from two to three. A substituted alkylene chain is a polymethylene group in which one or more methylene hydrogen atoms is replaced with a substituent.

Suitable substituents include those described below for a substituted aliphatic group. An alkylene chain also may be substituted at one or more positions with an aliphatic group or a substituted aliphatic group.

The term “alkenyl” refers to a straight or branched chain hydrocarbon containing from 2 to 12 carbons and containing at least one carbon-carbon double bond. Alkenyl groups can include C₂-C₁₂ alkenyl, C₂-C₁₀ alkenyl, C₂-C₆ alkenyl, C₂-C₅ alkenyl all of which are inclusive of C₄ alkenyls, C₃ alkenyls, and C₂ alkenyls. Non-limiting examples of alkenyl include ethenyl (vinyl), 1-propenyl, 2-propenyl (allyl), isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. In accordance with some example embodiments an alkenyl group can be optionally substituted.

The term “alkynyl” refers to a straight or branched chain hydrocarbon group containing from 2 to 12 carbon atoms and containing at least one carbon-carbon triple bond. Alkynyl groups can include C₂-C₁₂ alkynyl, C₂-C₁₀ alkynyl, C₂-C₆ alkynyl, C₂-C₅ alkynyl all of which are inclusive of C₄ alkynyl, C₃ alkynyl, and C₂ alkynyl. Non-limiting examples of alkynyl include, but are not limited, to acetylenyl (ethynyl), propynyl (i.e., 1-propynyl, 2-propynyl), butynyl, pentynyl, and the like. In accordance with some example embodiments an alkynyl group can be optionally substituted.

“Alkoxy” refers to a group of the formula-OR, where R is an alkyl, alkenyl, or alkynyl group, as defined herein, appended to the parent molecular moiety through the oxygen atom. Non-limiting examples of alkoxy groups include methoxy, ethoxy, propoxy, 2-propoxy, butoxy, tert-butoxy, pentyloxy, and hexyloxy. In accordance with some example embodiments an alkoxy group can be optionally substituted.

The term “aryl” refers to a stable monocyclic (i.e., phenyl), bicyclic, tricyclic or tetracyclic ring system containing 6 to 18 carbon atoms and at least one aromatic ring in the ring system. An aryl group can include fused and/or bridged ring systems. Non-limiting examples of aryl include aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In accordance with some example embodiments an aryl group can be optionally substituted.

The term “cycloalkyl” refers to a stable monocyclic, bicyclic, polycyclic, or spirocyclic fully saturated ring system typically comprising from 3 to 20 carbon atoms. Monocyclic ring systems are cyclic hydrocarbon groups that in some embodiments contain from 3 to 10 carbon atoms. Non-limiting examples of monocyclic cycloalkyls include cyclopropyl, cyclobutyl, cyclopentyl, cyclopentenyl, cyclohexyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Non-limiting examples of bicyclic and polycyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane, adamantyl, norbornyl, decalinyl, 7,7-dimethyl-bicyclo[2.2.1]heptanyl, and the like. In accordance with some example embodiments a cycloalkyl group can be optionally substituted.

“Cycloalkenyl” refers to a stable non-aromatic monocyclic, bicyclic, or polycyclic hydrocarbon consisting solely of carbon and hydrogen atoms, having one or more carbon-carbon double bonds, which can include fused or bridged ring systems, having from 3 to 20 carbon atoms, preferably having from 3 to 10 carbon atoms. Monocyclic cycloalkenyls include, for example, cyclopentenyl, cyclohexenyl, cycloheptenyl, cycloctenyl, and the like. Unless otherwise stated specifically in the specification, a cycloalkenyl group can be optionally substituted.

The term “halo” or “halogen” refers to one or a combination of —C₁, —Br, —I, or —F.

The terms “haloalkyl,” “haloalkenyl,” “haloalkynyl,” and “haloalkoxy” refer to an alkyl, alkenyl, alkynyl, or alkoxy group, as defined above, which is substituted with one or more halogen atoms at any available position. In accordance with some example embodiments any of these “halo-” groups can be optionally substituted.

The terms “heterocyclyl” and “heterocycle” refer to a 3- to 20-membered monocyclic, bicyclic, polycyclic, or spirocyclic ring system that may be saturated, unsaturated, or aromatic and that includes from 1 to 6 heteroatoms, N, O, or S. Monocyclic heterocycles include 3, 4, 5, 6, and 7 membered-rings containing at least 1 heteroatom independently selected from the group consisting of O, N, and S. The heterocycle is connected to the parent molecular moiety through any carbon atom or any nitrogen atom contained within the heterocycle. Non-limiting examples of monocyclic heterocycles include azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl(thiomorpholine sulfone), thiopyranyl, and trithianyl. Non-limiting examples of bicyclic heterocycles include 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl.

The term “oxo” as used herein means a ═O group.

The term “thia” as used herein means a ═S group.

The term “saturated” as used herein means the referenced chemical structure does not contain any multiple carbon-carbon bonds. For example, a saturated cycloalkyl group as defined herein includes cyclohexyl, cyclopropyl, and the like.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

When a range of values is listed, it is intended to encompass each value and sub-range within the range. For example, “C₁-C₆ alkyl” is intended to encompass C₁, C₂, C₃, C₄, C₅, C₆, C₁-6, C₁-5, C₁-4, C₁-3, C₁-2, C₂-6, C₂-5, C₂-4, C₂-3, C₃-6, C₃-5, C₃-4, C₄-6, C₄-5, and C₅-6 alkyl.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Both the R and the S stereochemical isomers, as well as all mixtures thereof, are included within the scope of the disclosure.

In view of the present disclosure, the methods and compositions described herein can be configured by the person skilled in the art to meet the desired need. In general, the disclosed materials and methods provide improvements in treatment of mental health disorders. The disclosed materials and methods also generally provide for improved agonists that are selective for particular 5-HT2 receptors.

### Compounds

In one aspect, the present disclosure provides compounds which act as agonists of the 5-HT2A receptor. In some embodiments, the compounds are full agonists of the 5-HT2A receptor. In some embodiments, the compounds are partial agonists of the 5-HT2A receptor. In some embodiments, the compounds display selectivity for the 5-HT2A receptor.

In some embodiments, the disclosure provides a compound of Formula (I):

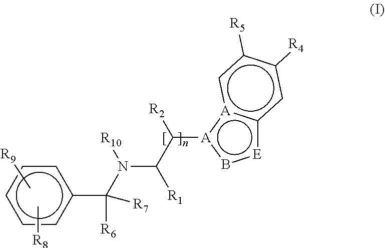

or a pharmaceutically acceptable salt thereof, wherein,
R₁, R₂, R₆, R₇, and independently are hydrogen, deuterium, alkyl, heteroalkyl, haloalkyl, cycloalkyl, or heterocyclyl;
R₄, R₅, R₈, and R₉ independently are hydrogen, deuterium, alkyl, heteroalkyl, haloalkyl, cycloalkyl, CO₂R₁₁, CONHR₁₁, CO₂N(R₁₁)(R₁₂), CN, or halogen,
   wherein R₁₁ and R₁₂ independently are hydrogen, deuterium, heteroalkyl, haloalkyl, cycloalkyl, or heterocyclyl;
R₁₀ is hydrogen, deuterium, or alkyl;
A is C or N;
B is CR₁₃, N, or CO,
   wherein R₁₃ is hydrogen, deuterium, alkyl, heteroalkyl, haloalkyl, cycloalkyl, or heterocyclyl;
E is CR₃, NR₃, N, O, or S;
   wherein R₃ is hydrogen, deuterium, alkyl, deuterated alkyl, heteroalkyl, haloalkyl, cycloalkyl, or heterocyclyl; and
n is 0 or 1.

Some embodiments comprise compounds of Formula (I) or a pharmaceutically acceptable salt thereof, wherein, R₁, R₂, R₃, R₆, and R₇ independently are hydrogen, deuterium, C₁-C₆ alkyl, C₁-C₆ heteroalkyl, C₁-C₆ haloalkyl, C₃-C₇ cycloalkyl, or C₃-C₇ heterocyclyl; R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, or CO₂N(R₁₁)(R₁₂); wherein R₁₁ and R₁₂ independently are C₁-C₆ heteroalkyl, C₁-C₆ haloalkyl, C₃-C₇ cycloalkyl, or C₃-C₇ heterocyclyl; R₁₀ is C₁-C₆ alkyl; B is CR₁₃; wherein R₁₃ is C₁-C₆ alkyl, C₁-C₆ heteroalkyl, C₁-C₆ haloalkyl C₃-C₇, cycloalkyl, or C₃-C₇ heterocyclyl.

In some embodiments, the compound of Formula (I) is a compound of Formula (I-A):

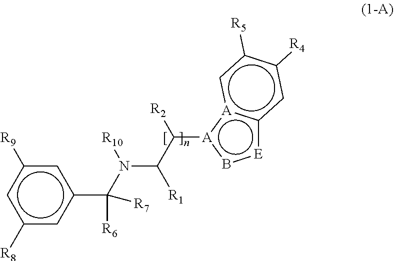

or a pharmaceutically acceptable salt thereof,
wherein,
R₁, R₂, R₆, R₇, and independently are hydrogen, deuterium, alkyl, heteroalkyl, haloalkyl, cycloalkyl, or heterocyclyl;
R₄, R₅, independently are hydrogen, deuterium, alkyl, deuterated alkyl, heteroalkyl, haloalkyl, cycloalkyl, CO₂R₁₁, CONHR₁₁, CO₂N(R₁₁)(R₁₂), CN, or halogen,
   wherein R₁₁ and R₁₂ independently are hydrogen, deuterium, heteroalkyl, haloalkyl, cycloalkyl, or heterocyclyl;
wherein when one A is N, R₈ and R₉ are not both hydrogen;
R₁₀ is hydrogen, deuterium, or alkyl;
A is C or N;
B is CR₁₃, N, or CO,
   wherein R₁₃ is hydrogen, deuterium, alkyl, heteroalkyl, haloalkyl, cycloalkyl, or heterocyclyl;
E is CR₃, NR₃, N, O, or S;
   wherein R₃ is hydrogen, deuterium, alkyl, deuterated alkyl, heteroalkyl, haloalkyl, cycloalkyl, or heterocyclyl; and
n is 0 or 1.

In some embodiments, the compound of Formula (I) is a compound of Formula (I-B):

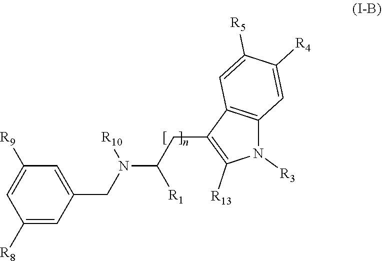

or a pharmaceutically acceptable salt thereof, wherein,
R₁ and R₃ independently are hydrogen, deuterium, C₁-C₆ alkyl, C₁-C₆ heteroalkyl, C₁-C₆ haloalkyl, C₃-C₇ cycloalkyl, or C₃-C₇ heterocyclyl;
R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, CO₂N(R₁₁)(R₁₂), CN, or halogen,
   wherein R₁₁ and R₁₂ independently are hydrogen, deuterium, C₁-C₆ heteroalkyl, C₁-C₆ haloalkyl, C₃-C₇ cycloalkyl, or C₃-C₇ heterocyclyl;
R₁₀ is hydrogen, deuterium, or C₁-C₆ alkyl;
R₁₃ is hydrogen, deuterium, C₁-C₆ alkyl, C₁-C₆ heteroalkyl, C₁-C₆ haloalkyl, C₃-C₇ cycloalkyl, or C₃-C₇ heterocyclyl; and
n is 0 or 1.

In some embodiments, the compound of Formula (I) is a compound of Formula (I-C):

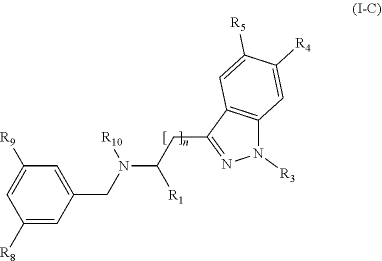

or a pharmaceutically acceptable salt thereof, wherein,
R₁ and R₃ independently are hydrogen, deuterium, C₁-C₆ alkyl, C₁-C₆ heteroalkyl, C₁-C₆ haloalkyl, C₃-C₇ cycloalkyl, or C₃-C₇ heterocyclyl;
R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, CO₂N(R₁₁)(R₁₂), CN, or halogen,
   wherein R₁₁ and R₁₂ independently are hydrogen, deuterium, C₁-C₆ heteroalkyl, C₁-C₆ haloalkyl, C₃-C₇ cycloalkyl, or C₃-C₇ heterocyclyl;
R₁₀ is hydrogen, deuterium, or C₁-C₆ alkyl;
n is 0 or 1.

In some embodiments, the compound of Formula (I) is a compound of Formula (I-D):

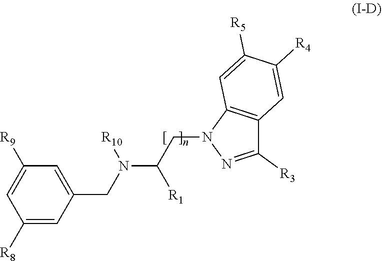

or a pharmaceutically acceptable salt thereof, wherein,
R₁ and R₃ independently are hydrogen, deuterium, C₁-C₆ alkyl, C₁-C₆ heteroalkyl, C₁-C₆ haloalkyl, C₃-C₇ cycloalkyl, or C₃-C₇ heterocyclyl;
R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, CO₂N(R₁₁)(R₁₂), CN, or halogen,
   wherein R₁₁ and R₁₂ independently are hydrogen, deuterium, C₁-C₆ heteroalkyl, C₁-C₆ haloalkyl, C₃-C₇ cycloalkyl, or C₃-C₇ heterocyclyl;
R₁₀ is hydrogen, deuterium, or C₁-C₆ alkyl;
n is 0 or 1.

In some embodiments, the compound of Formula (I) is a compound of Formula (I-E):

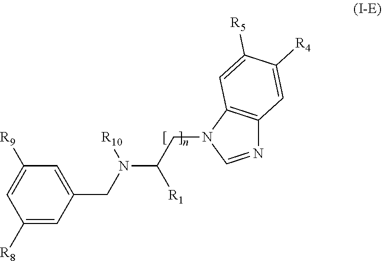

or a pharmaceutically acceptable salt thereof, wherein,
R₁ is hydrogen, deuterium, C₁-C₆ alkyl, C₁-C₆ heteroalkyl, C₁-C₆ haloalkyl, C₃-C₇ cycloalkyl, or C₃-C₇ heterocyclyl;
R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, CO₂N(R₁₁)(R₁₂), CN, or halogen,
   wherein R₁₁ and R₁₂ independently are hydrogen, deuterium, C₁-C₆ heteroalkyl, C₁-C₆ haloalkyl, C₃-C₇ cycloalkyl, or C₃-C₇ heterocyclyl;
R₁₀ is hydrogen, deuterium, or C₁-C₆ alkyl;
n is 0 or 1.

In some embodiments, the compound of Formula (I) is a compound of Formula (I-F):

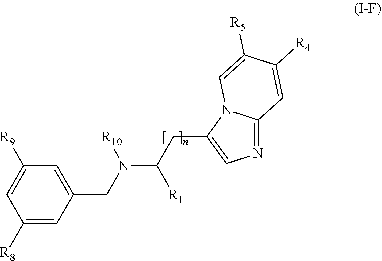

or a pharmaceutically acceptable salt thereof, wherein,
R₁ is hydrogen, deuterium, C₁-C₆ alkyl, C₁-C₆ heteroalkyl, C₁-C₆ haloalkyl, C₃-C₇ cycloalkyl, or C₃-C₇ heterocyclyl;
R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, CO₂N(R₁₁)(R₁₂), CN, or halogen,
   wherein R₁₁ and R₁₂ independently are hydrogen, deuterium, C₁-C₆ heteroalkyl, C₁-C₆ haloalkyl, C₃-C₇ cycloalkyl, or C₃-C₇ heterocyclyl;
R₁₀ is hydrogen, deuterium, or C₁-C₆ alkyl;
n is 0 or 1.

In some embodiments of the compounds of Formula (I), (I-A), (I-B), (I-C), (I-D), (I-E), or (I-F), R₁, R₂, R₃, R₆, and R₇ independently are hydrogen, deuterium, C₁-C₆ alkyl, C₁-C₆ heteroalkyl, C₁-C₆ haloalkyl, C₃-C₇ cycloalkyl, or C₃-C₇ heterocyclyl.

In some embodiments of the compounds of Formula (I), (I-A), (I-B), (I-C), (I-D), (I-E), or (I-F), R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, CO₂N(R₁₁)(R₁₂); wherein R₁₁ and R₁₂ independently are C₁-C₆ heteroalkyl, C₁-C₆ haloalkyl, C₃-C₇ cycloalkyl, or C₃-C₇ heterocyclyl.

In some embodiments of the compounds of Formula (I), (I-A), (I-B), (I-C), (I-D), (I-E), or (I-F), R₁₀ is C₁-C₆ alkyl.

In some embodiments of the compounds of Formula (I), (I-A), (I-B), (I-C), (I-D), (I-E), or (I-F), B is CR₁₃; wherein R₁₃ is C₁-C₆ alkyl, C₁-C₆ heteroalkyl, C₁-C₆ haloalkyl C₃-C₇, cycloalkyl, or C₃-C₇ heterocyclyl.

In some embodiments of the compounds of Formula (I), (I-A), (I-B), (I-C), (I-D), (I-E), or (I-F), n is 1.

In some embodiments of the compounds of Formula (I), (I-A), (I-B), (I-C), (I-D), (I-E), or (I-F), R₁ is hydrogen.

In some embodiments of the compounds of Formula (I), (I-A), (I-B), (I-C), (I-D), (I-E), or (I-F), R₁ is C₁-C₆ alkyl.

In some embodiments of the compounds of Formula (I), (I-A), (I-B), (I-C), (I-D), (I-E), or (I-F), R₁ is methyl.

In some embodiments of the compounds of Formula (I), (I-A), (I-B), (I-C), (I-D), (I-E), or (I-F), B is CR₁₃.

In some embodiments of the compounds of Formula (I), (I-A), (I-B), (I-C), (I-D), (I-E), or (I-F), B is CR₁₃, wherein R₁₃ is C₁-C₆ alkyl.

In some embodiments of the compounds of Formula (I), (I-A), (I-B), (I-C), (I-D), (I-E), or (I-F), B is CR₁₃, wherein R₁₃ is methyl.

In some embodiments of the compounds of Formula (I), (I-A), (I-B), (I-C), (I-D), (I-E), or (I-F), B is CR₁₃, wherein R₁₃ is hydrogen.

In some embodiments of the compounds of Formula (I), (I-A), (I-B), (I-C), (I-D), (I-E), or (I-F), B is CO.

In some embodiments of the compounds of Formula (I), (I-A), (I-B), (I-C), (I-D), (I-E), or (I-F), B is N.

In some embodiments of the compounds of Formula (I) or (I-A), one A is N.

In some embodiments of the compounds of Formula (I) or (I-A) one A is N and E is N.

In some embodiments of the compounds of Formula (I) or (I-A) one A is N and E is 0.

In some embodiments of the compounds of Formula (I), (I-A), (I-B), (I-C), (I-D), (I-E), or (I-F), E is CR₃ or NR₃, wherein R₃ is hydrogen.

In some embodiments of the compounds of Formula (I), (I-A), (I-B), (I-C), (I-D), (I-E), or (I-F), E is CR₃ or NR₃, wherein R₃ is C₁-C₆ alkyl.

In some embodiments of the compounds of Formula (I), (I-A), (I-B), (I-C), (I-D), (I-E), or (I-F), R₃ is methyl.

In some embodiments of the compounds of Formula (I), (I-A), (I-B), (I-C), (I-D), (I-E), or (I-F), R₃ is ethyl.

In some embodiments of the compounds of Formula (I), (I-A), (I-B), (I-C), (I-D), (I-E), or (I-F), R₄ and R₅ are hydrogen.

In some embodiments of the compounds of Formula (I), (I-A), (I-B), (I-C), (I-D), (I-E), or (I-F), R₄ is halogen.

In some embodiments of the compounds of Formula (I), (I-A), (I-B), (I-C), (I-D), (I-E), or (I-F), R₄ is I.

In some embodiments of the compounds of Formula (I), (I-A), (I-B), (I-C), (I-D), (I-E), or (I-F), R₅ is C₁-C₆ heteroalkyl.

In some embodiments of the compounds of Formula (I), (I-A), (I-B), (I-C), (I-D), (I-E), or (I-F), R₅ is methoxy.

In some embodiments of the compounds of Formula (I), (I-A), (I-B), (I-C), (I-D), (I-E), or (I-F), R₆ and R₇ are hydrogen.

In some embodiments of the compounds of Formula (I), (I-A), (I-B), (I-C), (I-D), (I-E), or (I-F), R₁ is hydrogen.

In some embodiments of the compounds of Formula (I), (I-A), (I-B), (I-C), (I-D), (I-E), or (I-F), R₁ is C₁-C₆ alkyl.

In some embodiments of the compounds of Formula (I), (I-A), (I-B), (I-C), (I-D), (I-E), or (I-F), R₁ is methyl.

In some embodiments of the compounds of Formula (I), (I-A), (I-B), (I-C), (I-D), (I-E), or (I-F), R₈ is C₁-C₆ alkyl.

In some embodiments of the compounds of Formula (I), (I-A), (I-B), (I-C), (I-D), (I-E), or (I-F), R₈ is methyl.

In some embodiments of the compounds of Formula (I), (I-A), (I-B), (I-C), (I-D), (I-E), or (I-F), R₉ is halogen.

In some embodiments of the compounds of Formula (I), (I-A), (I-B), (I-C), (I-D), (I-E), or (I-F), R₉ is Cl.

In some embodiments of the compounds of Formula (I), (I-A), (I-B), (I-C), (I-D), (I-E), or (I-F), R₉ is F.

In some embodiments of the compounds of Formula (I), (I-A), (I-B), (I-C), (I-D), (I-E), or (I-F), R₁₀ is C₁-C₆ alkyl.

In some embodiments of the compounds of Formula (I), (I-A), (I-B), (I-C), (I-D), (I-E), or (I-F), R₁₀ is methyl.

In some embodiments, the disclosure provides a compound of Formula (II):

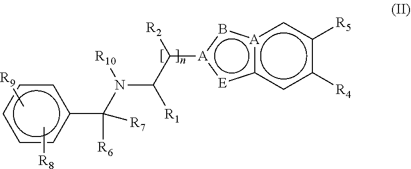

or a pharmaceutically acceptable salt thereof, wherein,
R₁, R₂, R₆, R₇, and independently are hydrogen, deuterium, alkyl, deuterated alkyl, heteroalkyl, haloalkyl, cycloalkyl, or heterocyclyl;
R₄, R₅, R₈, and R₉ independently are hydrogen, deuterium, alkyl, heteroalkyl, haloalkyl, cycloalkyl, CO₂R₁₁, CONHR₁₁, CO₂N(R₁₁)(R₁₂), CN, or halogen,
   Wherein R₁₁ and R₁₂ independently are hydrogen, deuterium, heteroalkyl, haloalkyl, cycloalkyl, or heterocyclyl;
R₁₀ is hydrogen, deuterium, or alkyl;
A is C or N;
B is CR₁₃, N, or CO,
   wherein R₁₃ is hydrogen, deuterium, alkyl, heteroalkyl, haloalkyl, cycloalkyl, or heterocyclyl;
E is CR₃, NR₃, N, O, or S;
   wherein R₃ is hydrogen, deuterium, alkyl, deuterated alkyl, heteroalkyl, haloalkyl, cycloalkyl, or heterocyclyl; and
n is 0 or 1.

Some embodiments comprise compounds of Formula (II) or a pharmaceutically acceptable salt thereof, wherein, R₁, R₂, R₃, R₆, and R₇ independently are hydrogen, deuterium, C₁-C₆ alkyl, C₁-C₆ heteroalkyl, C₁-C₆ haloalkyl, C₃-C₇ cycloalkyl, or C₃-C₇ heterocyclyl; R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, or CO₂N(R₁₁)(R₁₂); wherein R₁₁ and R₁₂ independently are C₁-C₆ heteroalkyl, C₁-C₆ haloalkyl, C₃-C₇ cycloalkyl, or C₃-C₇ heterocyclyl; R₁₀ is C₁-C₆ alkyl; B is CR₁₃; wherein R₁₃ is C₁-C₆ alkyl, C₁-C₆ heteroalkyl, C₁-C₆ haloalkyl C₃-C₇, cycloalkyl, or C₃-C₇ heterocyclyl.

In some embodiments, the compound of Formula (II) is a compound of Formula (II-A):

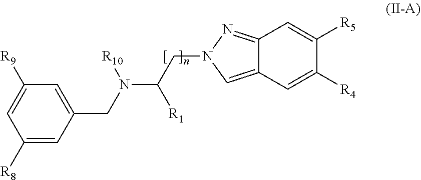

or a pharmaceutically acceptable salt thereof, wherein,
R₁ is hydrogen, deuterium, C₁-C₆ alkyl, C₁-C₆ heteroalkyl, C₁-C₆ haloalkyl, C₃-C₇ cycloalkyl, or C₃-C₇ heterocyclyl;
R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, CO₂N(R₁₁)(R₁₂), CN, or halogen,
   wherein R₁₁ and R₁₂ independently are hydrogen, deuterium, C₁-C₆ heteroalkyl, C₁-C₆ haloalkyl, C₃-C₇ cycloalkyl, or C₃-C₇ heterocyclyl;
R₁₀ is hydrogen, deuterium, or C₁-C₆ alkyl;
n is 0 or 1.

In some embodiments of the compounds of Formula (II) or (II-A), R₁, R₂, R₃, R₆, and R₇ independently are hydrogen, deuterium, C₁-C₆ alkyl, C₁-C₆ heteroalkyl, C₁-C₆ haloalkyl, C₃-C₇ cycloalkyl, or C₃-C₇ heterocyclyl.

In some embodiments of the compounds of Formula (II) or (II-A), R₄, R₅, R₈, and R₉ independently are hydrogen, deuterium, C₁-C₆ alkyl, C₁-C₆ heteroalkyl, C₁-C₆ haloalkyl, C₃-C₇ cycloalkyl, CO₂R₁₁, CONHR₁₁, or CO₂N(R₁₁)(R₁₂); wherein R₁₁ and R₁₂ independently are C₁-C₆ heteroalkyl, C₁-C₆ haloalkyl, C₃-C₇ cycloalkyl, or C₃-C₇ heterocyclyl.

In some embodiments of the compounds of Formula (II) or (II-A), R₁₀ is C₁-C₆ alkyl.

In some embodiments of the compounds of Formula (II) or (II-A), B is CR₁₃; wherein R₁₃ is C₁-C₆ alkyl, C₁-C₆ heteroalkyl, C₁-C₆ haloalkyl C₃-C₇, cycloalkyl, or C₃-C₇ heterocyclyl.

In some embodiments of the compounds of Formula (II) or (II-A), n is 1.

In some embodiments of the compounds of Formula (II) or (II-A), R₁ is hydrogen.

In some embodiments of the compounds of Formula (II) or (II-A), B is N.

In some embodiments of the compounds of Formula (II) or (II-A), one A is N.

In some embodiments of the compounds of Formula (II) or (II-A), E is CR₃ or NR₃, wherein R₃ is hydrogen.

In some embodiments of the compounds of Formula (II) or (II-A), R₄ and R₅ are hydrogen.

In some embodiments of the compounds of Formula (II) or (II-A), R₆ and R₇ are hydrogen.

In some embodiments of the compounds of Formula (II) or (II-A), R₈ is C₁-C₆ alkyl.

In some embodiments of the compounds of Formula (II) or (II-A), R₈ is methyl.

In some embodiments of the compounds of Formula (II) or (II-A), R₉ is halogen.

In some embodiments of the compounds of Formula (II) or (II-A), R₉ is Cl.

In some embodiments of the compounds of Formula (II) or (II-A), R₂ is hydrogen.

In some embodiments of the compounds of Formula (II) or (II-A), R₁₀ is hydrogen

In certain non-limiting example embodiments, the compounds can comprise those compounds illustrated in Table 1.

TABLE 1
Illustrative Compounds

| | |
|---|---|
| 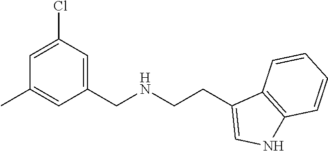 [SMILES: Cc1cc(Cl)cc(CNCCc2c[nH]c3ccccc23)c1] | 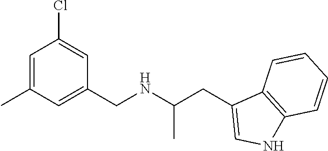 [SMILES: Cc1cc(Cl)cc(CNC(C)Cc2c[nH]c3ccccc23)c1] |
| 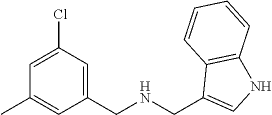 [SMILES: Cc1cc(Cl)cc(CNCc2c[nH]c3ccccc23)c1] | 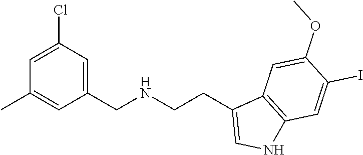 [SMILES: COc1cc2c(CCNCc3cc(C)cc(Cl)c3)c[nH]c2cc1I] |
| 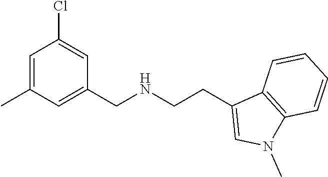 [SMILES: Cc1cc(Cl)cc(CNCCc2cn(C)c3ccccc23)c1] | 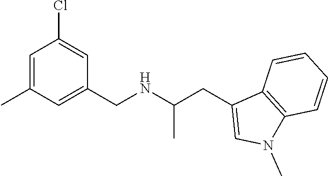 [SMILES: Cc1cc(Cl)cc(CNC(C)Cc2cn(C)c3ccccc23)c1] |
| 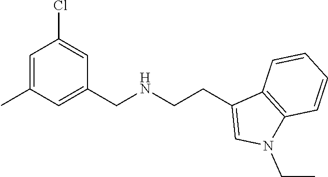 [SMILES: CCn1cc(CCNCc2cc(C)cc(Cl)c2)c2ccccc21] | 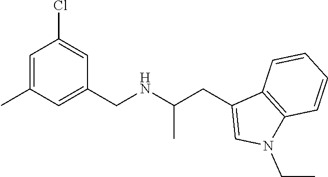 [SMILES: CCn1cc(CC(C)NCc2cc(C)cc(Cl)c2)c2ccccc21] |
| 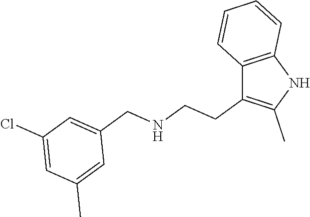 [SMILES: Cc1cc(Cl)cc(CNCCc2c(C)[nH]c3ccccc23)c1] | 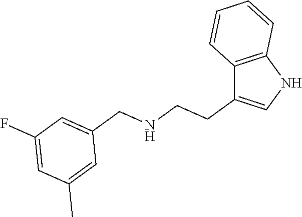 [SMILES: Cc1cc(F)cc(CNCCc2c[nH]c3ccccc23)c1] |
| 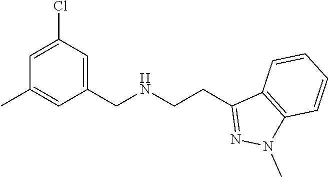 [SMILES: Cc1cc(Cl)cc(CNCCc2nn(C)c3ccccc23)c1] | 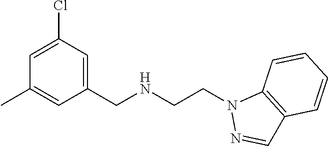 [SMILES: Cc1cc(Cl)cc(CNCCn2ncc3ccccc32)c1] |
| 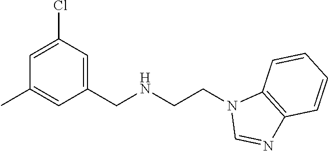 [SMILES: Cc1cc(Cl)cc(CNCCn2cnc3ccccc32)c1] | 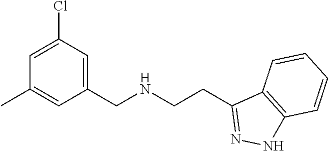 [SMILES: Cc1cc(Cl)cc(CNCCc2n[nH]c3ccccc23)c1] |
| 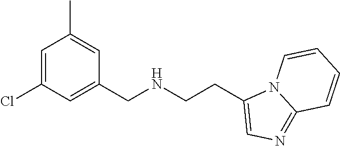 [SMILES: Cc1cc(Cl)cc(CNCCc2cnc3ccccn23)c1] | 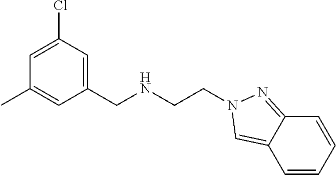 [SMILES: Cc1cc(Cl)cc(CNCCn2cc3ccccc3n2)c1] |
| 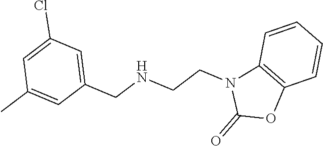 [SMILES: Cc1cc(Cl)cc(CNCCn2c(=O)oc3ccccc32)c1] | 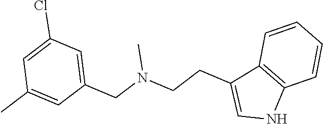 [SMILES: Cc1cc(Cl)cc(CN(C)CCc2c[nH]c3ccccc23)c1] |

In some embodiments, the compound of Formula (I), (I-A), (I-B), (I-C), (I-D), (I-E), or (I-F) is not:

| | |
|---|---|
| 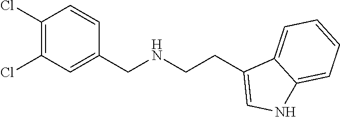 [SMILES: Clc1ccc(CNCCc2c[nH]c3ccccc23)cc1Cl] | 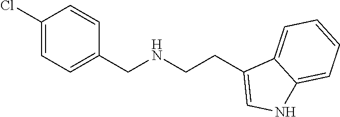 [SMILES: Clc1ccc(CNCCc2c[nH]c3ccccc23)cc1] |
| 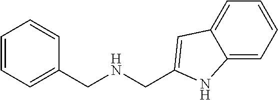 [SMILES: c1ccc(CNCc2cc3ccccc3[nH]2)cc1] | 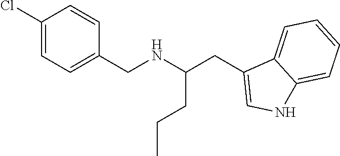 [SMILES: CCCC(Cc1c[nH]c2ccccc12)NCc1ccc(Cl)cc1] |
| 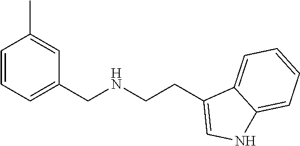 [SMILES: Cc1cccc(CNCCc2c[nH]c3ccccc23)c1] | 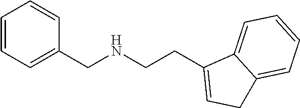 [SMILES: C1=C(CCNCc2ccccc2)c2ccccc2C1] |

### Salts

The compounds in accordance with the disclosure include pharmaceutically acceptable salts thereof, suitable for use in contact with the tissues of patients without undue toxicity, irritation, allergic response, and the like, commensurate with a reasonable benefit/risk ratio, and effective for their intended use, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.)

### Methods, Uses, and Therapeutics Applications

The compounds of the present disclosure find use, for example, in methods for modulating a serotonin receptor, e.g., 5-HT2A receptor. Accordingly, in some embodiments, the present disclosure provides the use of any one of compounds of the present disclosure (e.g., a compound of Formula (I), (I-A), (I-B), (I-C), (I-D), (I-E), (I-F), (II), (II-A), inclusive of the specific compounds described in Table 1, or a pharmaceutically acceptable salt thereof), for modulating serotonin receptor activity. Modulating serotonin (e.g., 5-HT2A) receptor activity can be in a subject in need thereof (e.g., a mammalian subject, such as a human) and for treatment of any of the described conditions or diseases herein, or those known to be associated with serotonin receptor activity. In some embodiments, modulating is activating or agonizing a serotonin receptor, e.g., 5-HT2A receptor. In some embodiments, the subject is a human.

In some embodiments, the present disclosure provides methods of treating a disease or disorder that is treatable by administration of a serotonin receptor agonist, e.g., 5-HT2A receptor agonist. In some embodiments, compounds in accordance with aspects and embodiments described herein have some amount of agonist activity for the 5-HT2A receptor (i.e., either partial agonist or full agonist of 5-HT2A). A full agonist will produce a maximal response when less than 100% of the receptors are occupied. A partial agonist will produce less than a maximal response even when 100% of the receptors are occupied.

In some embodiments the methods can be used to treat a neurological disease, and comprise administering a compound of the disclosure to a patient in need of treatment. As referred to herein, a “neurological disease” refers to any condition or disease involving the nervous system, for example, diseases that involve the central nervous system (brain, brainstem and cerebellum), the peripheral nervous system (including cranial nerves), and the autonomic nervous system. Within the broad scope of neurological diseases, a “neurodegenerative disease” refers to a neurological disease marked by the loss of nerve cells or damage to nerve cells, including non-limiting examples of Alzheimer's disease, Parkinson's disease, amyotrophic lateral sclerosis, tauopathies (including frontotemporal dementia), Huntington's disease, and the like. Further non-limiting examples of neurological diseases include headache, stupor and coma, dementia, seizure, sleep disorders, trauma, infections, neoplasms, neuro-ophthalmological conditions, movement disorders, demyelinating diseases, spinal cord disorders, disorders of peripheral nerves, muscle and neuromuscular junctions, among others. Addiction, mental illness, personality disorders, and rumination are also examples of neurological diseases and include a broad scope of conditions such as those discussed herein and as generally known in the art.

In some embodiments, the compounds are used in the treatment of pain (e.g., a painful condition) or a disease associated with pain. In embodiments, the use or method provides a form of pain management (e.g., reduce, eliminate, mitigate or relieve the symptoms). Non-liming examples of pain include neuropathic pain (e.g., peripheral neuropathic pain), central pain, deafferentation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. Any of the pain-related conditions can comprise one or more types of pain (e.g. nociceptive pain, inflammatory pain, neuropathic pain, etc.). In some embodiments, a particular source or type pain can dominate.

In certain embodiments, compounds are used in the treatment of a psychiatric disorder. The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D.C. (1994). Psychiatric disorders include anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

Accordingly, in some particular embodiments the disclosure provides for the use of the compounds of the disclosure in the treatment of one or more conditions including: dependence, addiction, and/or abuse of substances including, for example, alcohol, tobacco, nicotine, stimulants, and drugs (e.g., cocaine, cannabis, opioids); treatment of anxiety disorders, for example, post-traumatic stress disorder (PTSD), generalized anxiety disorder (GAD), obsessive-compulsive disorder (OCD), advanced-stage cancer-related anxiety, psychological distress (i.e., associated with existential crisis of terminal disease), and adjustment disorder with anxiety; treatment of depression, for example, cancer-related depression, treatment-resistant depression, major depressive disorder, severe existential depression; treatment of suicidality (i.e., ideation and actual attempt); treatment of demoralization including demoralization in older, long-term AIDS survivor men (OLTAS); treatment of pain, for example, cluster headaches, chronic pain, intractable phantom pain, or a disease associated with pain; treatment of personality disorders, for example, dysfunctional social cognition, maladaptive narcissism, borderline personality disorder (BPD), narcissistic personality disorder (NPD), psychopathy, emotional dysregulation and domestic violence/violence against one's partner; treatment of epilepsy; treatment of inflammation; and treatment of neurological diseases.

In some embodiments, the compounds of the present disclosure are used for treating a mental health disease or disorder in a subject in need thereof, the method comprising administering to the subject a therapeutically effective amount of a compound of the present disclosure (e.g., a compound of Formula (I), (I-A), (I-B), (I-C), (I-D), (I-E), (I-F), (II), (II-A), or inclusive of the specific compounds described in Table 1, or a pharmaceutically acceptable salt thereof) or a pharmaceutical composition comprising a compound of the present disclosure (e.g., a compound of Formula (I), (I-A), (I-B), (I-C), (I-D), (I-E), (I-F), (II), (II-A), or inclusive of the specific compounds described in Table 1, or a pharmaceutically acceptable salt thereof) and a pharmaceutically acceptable excipient.

In some embodiments, the mental health disease or disorder is depression, substance use disorders (SUD) or eating disorders.

In some embodiments, the mental health disease or disorder is an eating disorder. Eating disorders include illnesses such as anorexia nervosa, bulimia nervosa, and other disorders related to eating (e.g., binge eating).

In some embodiments, the mental health disease or disorder is a mood disorder. Mood disorders include e.g., depressive disorders, such as major depressive disorder or treatment resistant depression.

In some embodiments, the mental health disorder is a substance abuse disorder. In some embodiments, substance use related disorders are disorders of maladaptive patterns of substance use, and include criteria, such as recurrent substance use related problems, tolerance to a substance, withdrawal upon discontinuing use, an inability to cut down or control use of the substance, and giving up important social, occupational, or recreational activities because of using the substance. See e.g., the Diagnostic and Statistical Manual of Mental Disorders (DSM-5). In some embodiments, the substance use related disorder is a disorder resulting from the use of: alcohol; caffeine; cannabis; hallucinogens (such as phencyclidine or similarly acting arylcyclohexylamines, and other hallucinogens, such as LSD); inhalants; opioids; sedatives, hypnotics, or anxiolytics; stimulants (including amphetamine-type substances, cocaine, and other stimulants); tobacco; and other substances.

### Pharmaceutical Compositions

In some embodiments of the present disclosure, a pharmaceutical composition comprising a therapeutically effective amount of one or more compounds of the present disclosure (e.g., a compound of Formula (I), (I-A), (I-B), (I-C), (I-D), (I-E), (I-F), (II), (II-A), or inclusive of the specific compounds described in Table 1, or a pharmaceutically acceptable salt thereof) and a pharmaceutically acceptable excipient is provided.

For administration as compositions, including pharmaceutical compositions, the compounds are ordinarily combined with one or more carriers, diluents, and/or adjuvants appropriate for the indicated route of administration. The compounds may be mixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The compounds disclosed herein can be administered as the sole active pharmaceutical agent, or they can be used in combination with one or more other compounds useful for carrying out the methods and uses. When used or administered as a combination, the therapeutic agents can be formulated as separate compositions that are given at the same time or different times, or the therapeutic agents can be given as a single composition.

The compounds can be prepared in a solid form (including granules, powders or suppositories) or in a liquid form (e.g., solutions, suspensions, or emulsions). The disclosed compounds may be applied in a variety of solutions and may be subjected to conventional pharmaceutical operations such as sterilization and/or may contain conventional adjuvants, such as preservatives, stabilizers, wetting agents, emulsifiers, buffers, etc.

The disclosed compounds may be administered orally, topically, parenterally, by inhalation or spray or rectally in dosage unit formulations containing conventional non-toxic pharmaceutically acceptable carriers, adjuvants, and vehicles. The term parenteral as used herein includes percutaneous, subcutaneous, intravascular (e.g., intravenous), intramuscular, or intrathecal injection or infusion techniques and the like. One or more compounds in accordance with the disclosure may be present in association with one or more non-toxic pharmaceutically acceptable carriers and/or diluents and/or adjuvants, and if desired other active ingredients. Such pharmaceutical compositions may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions in accordance with the disclosure may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative, and flavoring and coloring agents. The pharmaceutical compositions may be in the form of a sterile injectable aqueous or oleaginous suspension.

This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds and pharmaceutical compositions in accordance with the disclosure may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds and pharmaceutical compositions in accordance with the disclosure may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The amount of compound(s) administered will depend upon a variety of factors, including, for example, the particular indication being treated, the mode of administration, whether the desired benefit is prophylactic or therapeutic, the severity of the indication being treated, the age and weight of the patient, the bioavailability of the particular compound(s), the metabolism rate and efficiency of the compound under the selected route of administration, etc. Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

The compound(s) described herein, or compositions thereof, will generally be used in an amount effective to achieve the intended result, for example in an amount effective to treat or prevent the particular disease being treated. By therapeutic benefit is meant eradication, delaying onset or progression, or amelioration of the underlying disorder being treated and/or eradication, delaying onset or progression, or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

### EXAMPLES

The preparation of the compounds of the disclosure (e.g., a compound of Formula (I), (I-A), (I-B), (I-C), (I-D), (I-E), (I-F), (II), (II-A), or Table 1) is illustrated by the following examples, which are not to be construed as limiting the disclosure in scope or spirit to the specific procedures and compounds described in them.

### General Experimental

In general, compounds of the present invention are prepared by the method illustrated in the general reaction scheme described below, or by modifications thereof, using readily available starting materials, reagents, and conventional synthetic procedures. However, those skilled in the art will recognize that other methods may also be suitable. Also, in these reactions, it is possible to make use of variants that are in themselves known but are not mentioned here. Unless otherwise noted, all materials/reagents were obtained from commercial suppliers and used without further purification. Reactions were monitored by LC-MS and/or thin layer chromatography (TLC) on silica gel 60 F254 (0.2 mm) pre-coated aluminum foil or glass-backed and visualized using UV light. 1HNMR (400 MHz) spectra was recorded on Bruker spectrometers at room temperature (RT) with TMS or residual solvent peak as internal standard. The line positions or multiples are given in (δ) and coupling constants (J) are given as absolute values in Hertz (Hz). The multiplicities in ¹HNMR spectra are abbreviated as follows: s (singlet), d (doublet), t (triplet), q (quartet), m (multiplet), br or broad (broadened). Preparative HPLC purification was performed on Shimadzu LC-6AD. All purification work was complete using Shim-pack PREP-DDS (H) KIT column. The mobile phases were water (with 0.1% HCO₂H) and acetonitrile. All reagents used were HPLC grade. The flow rate was 10 mL/min.

Preparative TLC was performed on Whatman LK6F Silica Gel 60A size 20×20 cm plates with a thickness of 1000 μm or equivalent. LC-MS was performed on Shimadzu LCMS-2020 equipped with LC-20AD or 30AD pumps, SPD-M20A PDA and Alltech 3300 ELSD. Mobile Phase A: water (0.1% formic acid); Mobile Phase B: acetonitrile (ACN); Duration: 5 minutes; Column: Sepax BR-C18 4.6*50 mm, 3 μm; Flow Rate: 1.0 mL/min; Oven Temperature: 40° C.

### Example 1: Synthesis of N-(3-chloro-5-methylbenzyl)-2-(1H-indol-3-yl) ethan-1-amine (1)

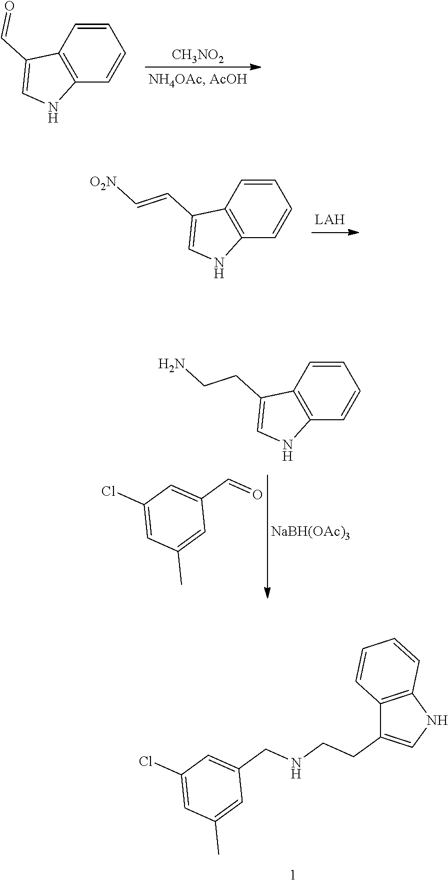

Step 1: Synthesis of 2-(1H-indol-3-yl) ethane-1-amine. A suspension of 1H-indole-3-carbaldehyde (0.5 g, 3.4 mmol, 1.0 eq), nitromethane (1.32 g, 17 mmol, 5.0 eq) and NH₄OAc (0.52 g, 8.5 mmol, 2.5 eq) in acetic acid (15 mL) was stirred at 60° C. overnight. The reaction was monitored by TLC until the reaction was complete. The resulting mixture was poured into water (20 mL) and a yellow solid appeared. The yellow solid was filtered and dried at 50° C. to afford 3-(2-nitrovinyl)-1H-indole (200 mg, 47%) as a yellow solid. ¹H NMR (400 MHz, Solvent: DMSO) ppm 12.24 (s, 1H), 8.34-8.39 (d, J=16 Hz, 1H), 8.25 (s, 1H), 8.03-7.99 (d, J=16 Hz, 1H), 7.97-7.95 (d, J=8 Hz, 1H), 7.53-7.51 (d, J=8 Hz, 1H), 7.30-7.22 (m, 2H). The crude product was used without further purification. 3-(2-nitrovinyl)-1H-indole (200 mg, 1.06 mmol, 1.0 eq) dissolved in MeCN (5 mL) and added to a suspension of LiAlH₄ (161 mg, 4.0 eq) in THF (5 mL) at 0° C. The mixture was stirred at RT overnight. The reaction was monitored by TLC until the reaction was complete. Water (3 mL) was added followed by addition of NaOH/H₂O (9 mL). The resulting mixture was extracted with DCM (15 mL×3). The combined organic layers were dried over Na₂SO₄ and concentrated under reduced pressure to give a crude product which was purified by column chromatography (eluting with DCM/MeOH=10:1) to afford the title compound (100 mg, 54%) as an oil. LCMS: m/z 161 [M+H]⁺. ¹H NMR (400 MHz, Solvent: CDCl₃) ppm 8.06 (s, 1H), 7.63-7.61 (d, J=8 Hz, 1H), 7.38-7.36 (d, J=8 Hz, 1H), 7.22-7.20 (t, J=7.2 Hz, 1H), 7.14-7.12 (t, J=7.2 Hz, 1H), 7.049-7.

Step 2: Synthesis of N-(3-chloro-5-methylbenzyl)-2-(1H-indol-3-yl) ethan-1-amine (1). To a stirred solution of 2-(1H-indol-3-yl) ethan-1-amine (100 mg, 0.64 mmol, 1.0 eq) and 3-chloro-2-methylbenzaldehyde (96 mg, 0.64 mmol, 1.0 eq) in dichloromethane (8 mL) was added NaBH(OAc)₃ (661 mg, 3.12 mmol, 5.0 eq). The resulting mixture was stirred at RT overnight. The reaction mixture was quenched with aqueous sodium bicarbonate solution and extracted with ethyl acetate. The combined organic layers were dried over anhydrous sodium sulfate and concentrated under reduced pressure to give a crude residue which was purified by flash chromatography (eluted with petroleum ether/EA=10:1 to 1:1 gradient) to afford the title compound (100 mg, 54%) as a colorless oil. LCMS: m/z 299 [M+H]⁺. ¹H NMR (400 MHz, Solvent: CDCl₃) ppm 8.01 (s, 1H), 7.61 (d, J=7.8 Hz, 1H), 7.36 (d, J=8.1 Hz, 1H), 7.20 (t, J=7.2 Hz, 1H), 7.12 (t, J=7.2 Hz, 1H), 7.07 (s, 1H), 7.05-7.01 (m, 2H), 6.94 (s, 1H), 3.74 (s, 2H), 3.04-2.93 (m, 4H), 2.27 (s, 3H).

### Example 2: Synthesis of N-(3-chloro-5-methylbenzyl)-2-(1H-indol-3-yl) ethan-1-amine hydrochloride (2)

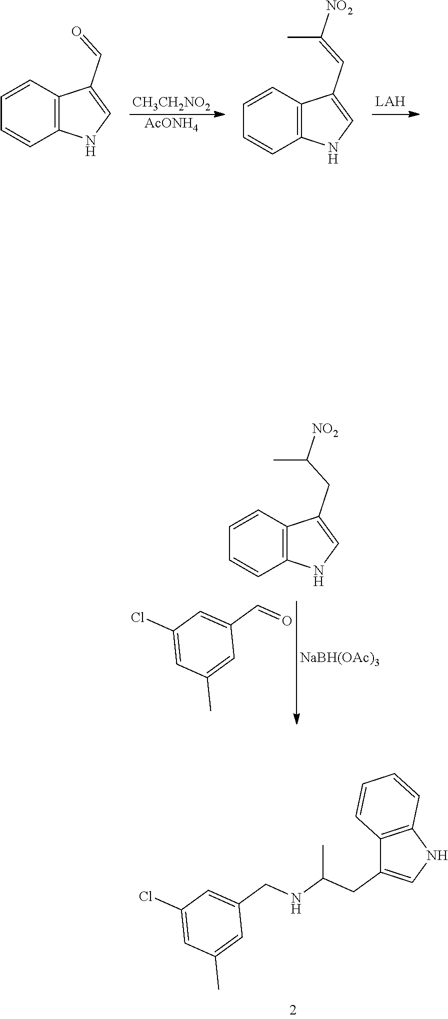

Step 1: Synthesis of 3-(2-nitroprop-1-en-1-yl)-1H-indole. The same procedure as for the synthesis of 1 was applied to prepare 2 (3 g, 74% yield) as a yellow solid. ¹H NMR (400 MHz, Solvent: CDCl₃) δ 8.53 (s, 1H), 7.83 (d, J=7.7 Hz, 1H), 7.57 (d, J=2.8 Hz, 1H), 7.47 (d, J=7.7 Hz, 1H), 7.38-7.28 (m, 2H), 2.55 (s, 3H).

Step 2: Synthesis of 1-(1H-indol-3-yl) propan-2-amine. The same procedure as for the synthesis of 1 was applied (130 mg, 30%) as a white solid. LCMS: m/z 175 [M+H]⁺. ¹H NMR (400 MHz, Solvent: CDCl₃) δ 7.62 (d, J=7.9 Hz, 1H), 7.37 (d, J=8.1 Hz, 1H), 7.20 (t, J=7.4 Hz, 1H), 7.12 (t, J=7.4 Hz, 1H), 7.05 (d, J=1.8 Hz, 1H), 3.30 (dd, J=5.1, 1.8 Hz, 1H), 2.89 (dd, J=14.1, 4.9 Hz, 1H), 2.66 (dd, J=14.2, 8.3 Hz, 1H), 1.17 (d, J=6.3 Hz, 3H).

Step 3: Synthesis of N-(3-chloro-5-methylbenzyl)-1-(1H-indol-3-yl) propan-2-amine. The same procedure as for the synthesis of 1 was applied to afford the title compound which was converted to the HCl salt (52 mg, 25%) as a white solid after treatment with HCl in dioxane. LCMS: m/z 314 [M+H]⁺. ¹H NMR (400 MHz, Solvent: CDCl₃) ppm 11.02 (s, 1H), 9.27 (dd, J=2.3, 1.3 Hz, 2H), 7.54 (d, J=8.9 Hz, 2H), 7.37 (dd, J=12.8, 4.6 Hz, 3H), 7.26 (d, J=1.8 Hz, 1H), 7.09 (t, J=7.5 Hz, 1H), 7.00 (t, J=7.4 Hz, 1H), 4.24 (d, J=4.5 Hz, 2H), 3.37 (d, J=14.6 Hz, 2H), 2.85 (dd, J=13.7, 10.1 Hz, 1H), 2.34 (s, 3H), 1.22 (d, J=6.4 Hz, 3H).

### Example 3: Synthesis of N-((1H-indol-3-yl)methyl)-1-(3-chloro-5-methylphenyl) methanamine hydrochloride (3)

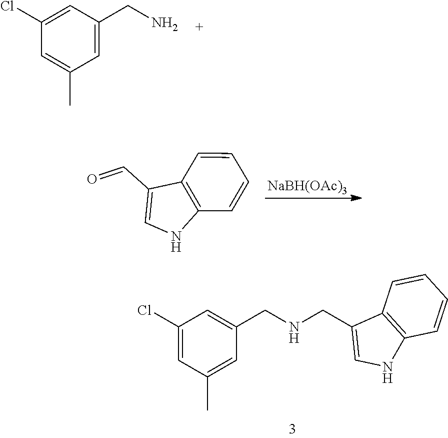

The same procedure as for the synthesis of 1 was applied to afford the title compound as a white solid (46 mg, 82%). LCMS: (ES−): m/z 285.2 [M+H]⁺. ¹H NMR (400 MHz, Solvent: DMSO-d6) ppm 11.4 (s, 1H), 9.50-9.40 (m, 2H), 7.68 (d, J=8.0 Hz, 1H), 7.59 (d, J=2.4 Hz, 1H), 7.46 (s, 1H), 7.43 (d, J=7.2 Hz, 1H), 7.15 (t, J=14.8 Hz, 7.6 Hz, 1H), 7.08 (t, J=14.8 Hz, 7.6 Hz, 1H), 4.31 (d, J=4.0 Hz, 1H), 4.14 (d, J=4.0 Hz, 1H), 2.31 (s, 3H).

### Example 4: Synthesis of N-(3-chloro-5-methylbenzyl)-2-(6-iodo-5-methoxy-1H-indol-3-yl) ethan-1-amine (4)

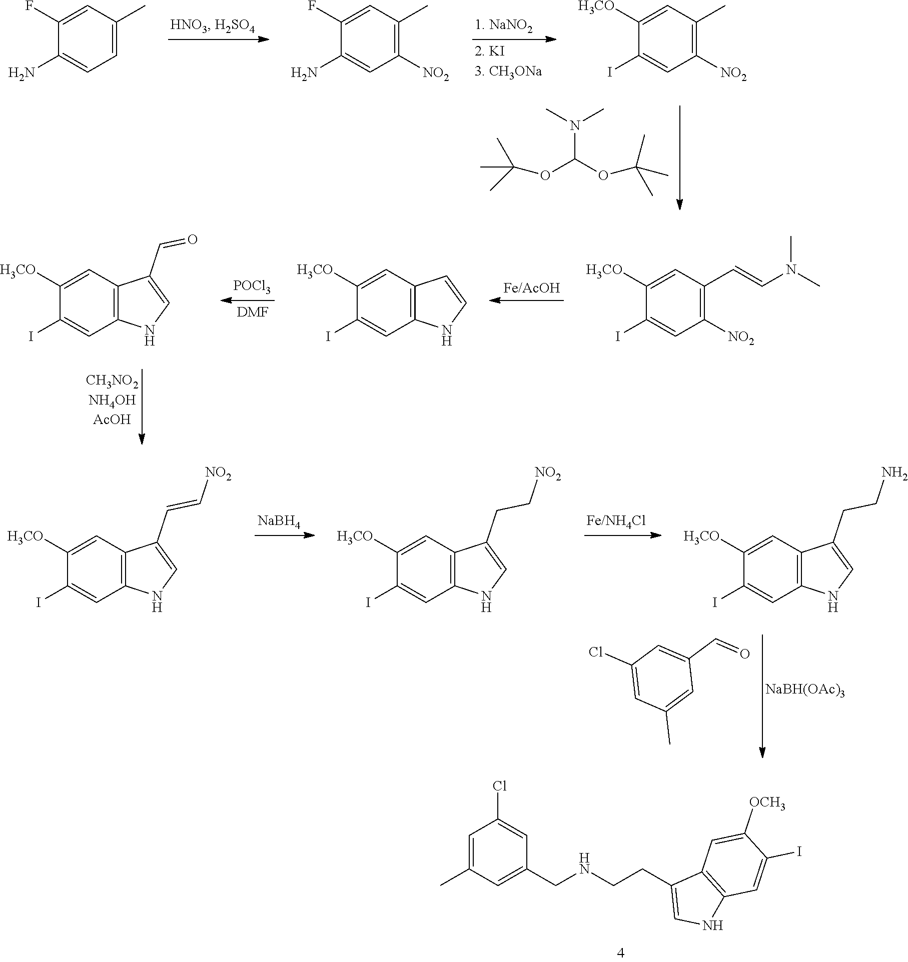

Step 1: Synthesis of 2-fluoro-4-methyl-5-nitroaniline. A suspension of 2-fluoro-4-methylaniline (2 g, 16 mmol, 1 eq) in H₂SO₄ (30 g) was stirred at −10° C. and HNO₃ (5 mL) was added slowly. The resulting mixture was stirred at −10° C. for 30 min. The mixture was poured into ice water (150 mL) and the resulting mixture was extracted with EtOAc (30 mL×5). The combined organic extracts were collected and concentrated under reduced pressure to afford the title compound (1.6 g). ¹H NMR (400 MHz, Solvent: DMSO-d6) ppm 7.45 (d, J=12.0 Hz, 1H), 7.18 (d, J=12.0 Hz, 1H), 6.80-6.53 (br, 3H), 2.37 (s, 3H).

Step 2: Synthesis of 1-fluoro-2-iodo-5-methyl-4-nitrobenzene. To a solution of 2-fluoro-4-methyl-5-nitroaniline (0.8 g, 4.7 mmol, 1 eq) dissolved in 4 M HCl (10 mL) was added NaNO₂ (350 mg, 5.17 mmol 1.1 eq) at 0° C. The mixture was stirred at 0° C. for 0.5 h followed by the addition of KI (2.73 g, 16.4 mmol, 3.5 eq). The resulting mixture was stirred at RT overnight. The reaction was extracted by EtOAc (30 mL×3). The combined organic extracts were collected and concentrated under reduced pressure. The residue was purified by flash chromatography to afford the title compound (0.84 g). ¹H NMR (400 MHz, Solvent: CDCl₃) ppm 8.43 (d, J=5.6 Hz, 1H), 7.03 (d, J=8.0 Hz, 1H), 2.59 (s, 3H).

Step 3: Synthesis of 1-iodo-2-methoxy-4-methyl-5-nitrobenzene. To a stirred solution of 1-fluoro-2-iodo-5-methyl-4-nitrobenzene (400 mg, 1.42 mmol, 1.0 eq) in MeOH (4 mL) was added 30% MeONa/MeOH (0.27 g, 1.5 mmol, 1.1 eq). The resulting mixture was stirred at RT overnight. The resulting mixture was washed with water (20 mL), extracted with EtOAc (30 mL×3). The combined organic extracts were collected and concentrated under reduced pressure to afford the title compound (320 mg). ¹H NMR (400 MHz, Solvent: CDCl₃) ppm 8.52 (s, 1H), 6.66 (s, 1H), 3.96 (s, 3H), 2.63 (s, 3H).

Step 4: Synthesis of (E)-2-(4-iodo-5-methoxy-2-nitrophenyl)-N,N-dimethylethen-1-amine. To a stirred solution of 1-iodo-2-methoxy-4-methyl-5-nitrobenzene (5.5 g, 18.7 mmol, 1.0 eq) in DMF (40 mL) was added 1,1-di-tert-butoxy-N,N-dimethylmethanamine (10 mL). The resulting mixture was stirred at 135° C. overnight. The resulting mixture was washed with water (400 mL), and extracted with EtOAc (50 mL×4). The combined organic extracts were collected and concentrated under reduced pressure. The resulting residue was used in the next step without purification.

Step 5: Synthesis of 6-iodo-5-methoxy-1H-indole. A solution of (E)-2-(4-iodo-5-methoxy-2-nitrophenyl)-N,N-dimethylethen-1-amine (2 g, 5.7 mmol, 1.0 eq) and Fe (1.2 g, 22 mmol, 4 eq) in AcOH (50 mL) was heated at 90° C. for 2 h. The resulting mixture was washed by water (400 mL), extracted with EtOAc (50 mL×4). The combined organic extracts were collected and concentrated under reduced pressure. The resulting residue was purified by flash chromatography to afford the title compound (600 mg). ¹H NMR (400 MHz, Solvent: CDCl₃) ppm 8.17 (br, 1H), 7.81 (d, J=4.0 Hz, 1H), 7.15 (t, J=2.8 Hz, 1H), 7.09 (s, 1H), 6.46 (t, J=2.4 Hz, 1H), 3.91 (s, 3H). Step 6: Synthesis of 6-iodo-5-methoxy-1H-indole-3-carbaldehyde. A solution of 6-iodo-5-methoxy-1H-indole (100 mg, 0.36 mmol, 1.0 eq) in POCl₃ (5 mL) was added dry DMF (10 mL). The resulting mixture was stirred at RT overnight. The resulting mixture was washed by water (400 mL) and extracted with EtOAc (50 mL×4). The combined organic extracts were collected and concentrated under reduced pressure. The resulting residue was purified by flash chromatography to afford the title compound (100 mg). ¹H NMR (400 MHz, Solvent: DMSO-d₆) ppm 12.03 (s, 1H), 9.91 (s, 1H), 8.23 (d, J=2.8 Hz, 1H), 7.92 (s, 1H), 7.62 (s, 1H), 3.85 (s, 3H).

Step 7: Synthesis of (E)-6-iodo-5-methoxy-3-(2-nitrovinyl)-1H-indole. A solution of 6-iodo-5-methoxy-1H-indole-3-carbaldehyde (100 mg, 0.33 mmol, 1.0 eq.) in CH₃NO₂ (5 mL) was added dry NH₄OAc (60 mg). The resulting mixture was stirred at RT overnight. The resulting mixture was washed with water (400 mL) and extracted with EtOAc (50 mL×4). The combined organic extracts were collected and concentrated under reduced pressure. The resulting residue was purified by flash chromatography to afford the title compound (100 mg). ¹H NMR (400 MHz, Solvent: DMSO-d₆) ppm 12.10 (s, 1H), 8.43 (d, J=13.6 Hz, 1H), 8.19 (s, 1H), 8.11 (d, J=13.6 Hz, 1H), 7.91 (s, 1H), 7.46 (s, 1H), 3.93 (s, 3H).

Step 8: Synthesis of 6-iodo-5-methoxy-3-(2-nitroethyl)-1H-indole. A solution of (E)-6-iodo-5-methoxy-3-(2-nitrovinyl)-1H-indole (484 mg, 1.4 mmol, 1.0 eq.) in THF (5 mL) and MeOH (5 mL) was added NaBH₄ (100 mg, 2.8 mmol, 2.0 eq.). The resulting mixture was stirred at RT overnight. The resulting mixture was washed by water (400 mL) and extracted by EtOAc (50 mL×4). The combined organic extracts were collected and concentrated under reduced pressure. The resulting residue was purified by flash chromatography to afford the title compound. (253 mg). ¹H NMR (400 MHz, Solvent: DMSO-d₆) ppm 10.82 (s, 1H), 7.47 (s, 1H), 7.20 (s, 1H), 7.16 (d, J=2.4 Hz, 1H), 4.82 (t, J=7.4 Hz, 2H), 3.83 (s, 3H), 3.30 (s, 2H).

Step 9: Synthesis of 2-(6-iodo-5-methoxy-1H-indol-3-yl) ethan-1-amine. A solution of 6-iodo-5-methoxy-3-(2-nitroethyl)-1H-indole (253 mg, 0.73 mmol, 1.0 eq) in EtOH (10 mL) and was added Fe (162 mg, 2.9 mmol, 4 eq) and NH₄Cl (154 mg, 2.9 mmol, 4 eq). The resulting mixture was stirred at 80° C. overnight. The resulting mixture was filtered. The filter cake was washed by EtOAc twice. The combined organic washes were collected and concentrated under reduced pressure. The residue was purified by flash chromatography to afford the title compound (193 mg). ¹H NMR (400 MHz, MeOD) δ 7.79 (s, 1H), 7.14 (s, 1H), 7.11 (s, 1H), 3.88 (s, 3H), 3.22 (t, J=7.3 Hz, 2H), 3.08 (t, J=7.3 Hz, 2H).

Step 10: Synthesis of N-(3-chloro-5-methylbenzyl)-2-(6-iodo-5-methoxy-1H-indol-3-yl) ethan-1-amine (4). A solution of 2-(6-iodo-5-methoxy-1H-indol-3-yl) ethan-1-amine (193 mg, 0.61 mmol, 1.0 eq) in DCM (10 mL) and was added 3-chloro-5-methylbenzaldehyde (103 mg, 0.67 mmol, 1.1 eq) and 1 drop of AcOH. The resulting mixture was stirred at RT for 0.5 h. followed by the addition of NaBH(OAc)₃ (636 mg, 1.83 mmol, 3.0 eq). The resulting mixture was stirred at RT overnight. The resulting mixture was washed with NaHCO₃ (aq) and extracted by EtOAc (50 mL×3). The combined organic extracts were collected and concentrated under reduced pressure. The resulting residue was purified by flash chromatography to afford the title compound (174 mg). ¹H NMR (400 MHz, MeOD) δ 7.65 (s, 1H), 6.98 (s, 1H), 6.96 (s, 1H), 6.93 (s, 1H), 6.89 (s, 1H), 6.82 (s, 1H), 3.71 (s, 3H), 3.62 (s, 2H), 2.80 (dd, J=16.1, 6.2 Hz, 4H), 2.15 (s, 3H).

### Example 5: Synthesis of N-(3-chloro-5-methylbenzyl)-2-(1-methyl-1H-indol-3-yl) ethan-1-amine (5)

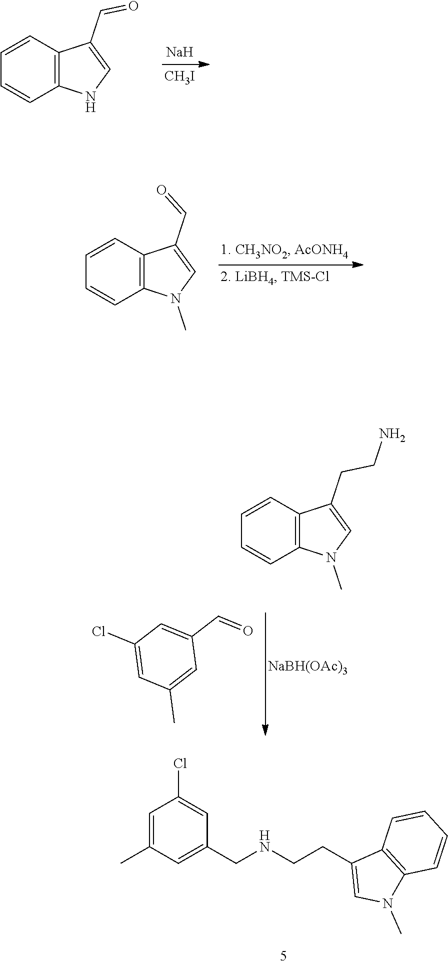

Step 1: Synthesis of 1-methyl-1H-indole-3-carbaldehyde. A suspension of 1H-indole-3-carbaldehyde (2 g, 14 mmol, 1.0 eq) and NaH (0.98 g, 41 mmol, 3.0 eq) in DMF (15 mL) was stirred at RT for 30 min under N₂. CH₃I (2.9 g, 21 mmol, 1.5 eq) was added to the above mixture slowly. The resulting mixture was stirred at RT overnight. The resulting mixture was extracted with EA (50 mL×3). The combined organic extracts were dried over anhydrous sodium sulfate and concentrated under reduced pressure to give a crude residue which was purified by flash chromatography (eluted with petroleum/EA=5:1) to afford the title compound (1.9 g oil, 87%). ¹H NMR (400 MHz, CDCl₃) δ 9.98 (s, 1H), 8.30 (dd, J=6.5, 2.1 Hz, 1H), 7.65 (s, 1H), 7.37-7.29 (m, 3H), 3.85 (s, 3H).

Step 2: Synthesis of (E)-1-methyl-3-(2-nitrovinyl)-1H-indole. A suspension of 1-methyl-1H-indole-3-carbaldehyde (1 g, 6 mmol, 1.0 eq), nitromethane (1.9 g, 31. mmol, 5.0 eq) and ammonium acetate (0.75 g, 31 mmol, 5.0 eq) in 10 mL acetic acid was heated to 60° C. overnight. A yellow solid formed. The reaction was cooled to RT, 40 mL water and 3 mL EtOH was added and the resulting mixture was stirred 30 min. The resulting mixture was filtered, washed with water (10 mL×3) and dried at 50° C. to afford the title compound 1.1 g (87%) as a yellow solid. LCMS: m/z no detected. ¹H NMR (400 MHz, CDCl₃) δ 8.26 (d, J=13.4 Hz, 1H), 7.82-7.72 (m, 2H), 7.52 (s, 1H), 7.45-7.31 (m, 3H), 3.88 (d, J=4.7 Hz, 3H).

Step 3: Synthesis of 2-(1-methyl-1H-indol-3-yl) ethan-1-amine. A suspension of LiBH₄ (64 mg, 2.97 mmol, 4.0 eq) in 15 mL THF was cooled to 0° C. under N₂. TMSCl (641 g, 5.93 mmol, 8.0 eq) was added to the solution slowly at 0° C. After the addition was complete, the resulting mixture was stirred for 30 min at 0° C. (E)-1-methyl-3-(2-nitrovinyl)-1H-indole (150 mg, 0.74 mmol, 1.0 eq) dissolved in 5 mL THF was added and the resulting mixture was heated at reflux for 4 h. The reaction was monitored by TLC until the reaction was complete. Water (20 mL) was added to quench the reaction followed by Na₂CO₃ (aq.) was added to adjust pH to 11. The resulting mixture was extracted with DCM (15 mL×3). The combined organic extracts were died over Na₂SO₄ and concentrated under reduced pressure to afford a crude residue which was purified by flash chromatography (eluted with DCM/CH₃OH=20:1) to afford the title compound (80 mg, 62%) as an oil. LCMS: m/z 175 [M+H]⁺. ¹H NMR (400 MHz, DMSO) δ 7.59 (d, J=7.8 Hz, 1H), 7.40 (d, J=8.2 Hz, 1H), 7.22 (s, 1H), 7.19-7.12 (m, 1H), 7.08-7.00 (m, 1H), 3.74 (s, 3H), 3.02 (s, 4H).

Step 4: Synthesis of N-(3-chloro-5-methylbenzyl)-2-(1-methyl-1H-indol-3-vl) ethan-1-amine (5). To a stirred solution of 2-(1-methyl-1H-indol-3-yl) ethan-1-amine (80 mg, 0.39 mmol, 1.0 eq.) and 3-chloro-5-methyl benzaldehyde (60 mg, 0.39 mmol, 1.0 eq.) in dichloromethane (20 mL) was added NaBH(OAc)₃ (415 mg, 1.96 mmol, 5.0 eq.). The resulting mixture was stirred at RT overnight. The reaction mixture was quenched with aqueous sodium bicarbonate solution and extracted with ethyl acetate. The organic layer was dried over anhydrous sodium sulfate and concentrated under reduced pressure to give a crude residue which was purified by flash chromatography (eluted with petroleum ether/EA=1:1) to afford the title compound which was converted to the HCl salt (40 mg, 29% yield) as white solid after treatment with HCl in dioxane. LCMS: m/z 313.10 [M+H]⁺. ¹H NMR (400 MHz, DMSO) δ 9.41 (s, 2H), 7.58 (d, J=7.8 Hz, 1H), 7.50 (s, 1H), 7.41 (d, J=8.2 Hz, 1H), 7.34 (d, J=8.7 Hz, 2H), 7.21 (s, 1H), 7.19-7.13 (m, 1H), 7.08-7.02 (m, 1H), 4.15 (t, J=5.6 Hz, 2H), 3.74 (s, 3H), 3.12 (s, 4H), 2.33 (s, 3H).

### Example 6: Synthesis of N-(3-chloro-5-methylbenzyl)-1-(1-methyl-1H-indol-3-yl) propan-2-amine (6)

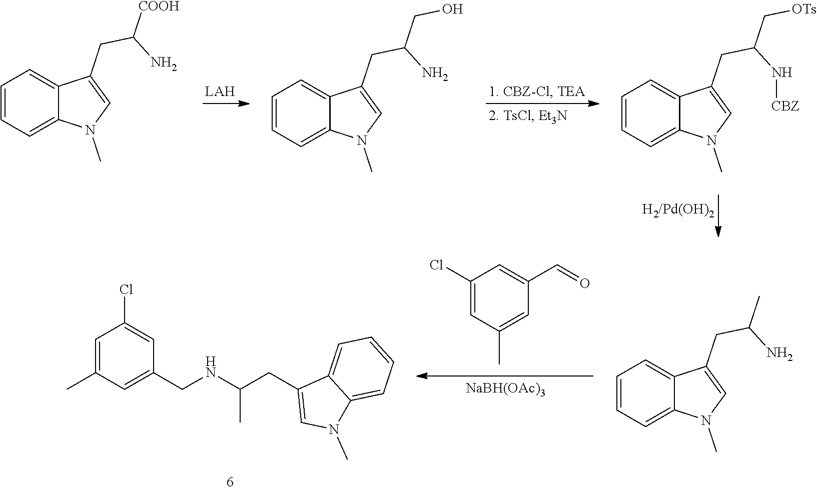

Step 1: Synthesis of 2-amino-3-(1-methyl-1H-indol-3-yl) propan-1-ol. A suspension of LiAlH₄ (0.69 g, 18.3 mmol, 4 eq) in THF (5 mL) was stirred at 0° C. under N₂. 1-methyltryptophan (1 g, 4.5 mmol, 1 eq) in 3 mL THF was added slowly to the above mixture. The resulting mixture was heated at reflux and stirred overnight. Water (200 mL) was added to quench the reaction. The resulting mixture was extracted with DCM (50 mL×3). The combined organic extracts were dried over anhydrous sodium sulfate and concentrated under reduced pressure to give a crude product (500 mg) which was used in the next step without additional purification.

Step 2: Synthesis of 2benzyl (1-hydroxy-3-(1-methyl-1H-indol-3-yl) propan-2-yl)carbamate. A suspension of 2-amino-3-(1-methyl-1H-indol-3-yl) propan-1-ol (500 mg, 24.4 mmol, 1 eq) and TEA (1.23 g, 12.2 mmol, 5 eq) in DCM (10 mL) was cooled to 0° C. CbzCl (417 mg, 24.4 mmol, 1 eq) was added slowly. The resulting mixture was stirred at RT overnight. The organic phase was concentrated under reduced pressure to afford a crude residue which was purified by flash chromatography (eluted with DCM/CH₃OH=50:1) to afford the title compound (250 mg, 30%) as an oil. LCMS: m/z 339 [M+H]⁺.

Step 3: Synthesis of 2-(((benzyloxy) carbonyl)amino)-3-(1-methyl-1H-indol-3-yl) propyl 4-methylbenzenesulfonate. A suspension of 2 benzyl (1-hydroxy-3-(1-methyl-1H-indol-3-yl) propan-2-yl)carbamate (250 mg, 0.73 mmol, 1 eq) and TEA (188 mg, 1.46 mmol, 2 eq) in DCM (10 mL) was cooled to 0° C. TsCl (149 mg, 0.78 mmol, 1 eq) was added to the above mixture slowly. The resulting mixture was stirred at RT overnight. The organic phase was concentrated under reduced pressure to afford a crude residue which was purified by flash chromatography (eluted with petroleum ether/EA=4:1) to afford the title compound (145 mg, 39%) as an oil. LCMS: m/z 493 [M+H]⁺.

Step 4: Synthesis of 1-(1-methyl-1H-indol-3-yl) propan-2-amine. A suspension of 2-(((benzyloxy)carbonyl)amino)-3-(1-methyl-1H-indol-3-yl) propyl 4-methylbenzenesulfonate (145 mg, 0.29 mmol, 1 eq) and Pd(OH)₂ (15 mg, 10%) in EtOH (5 mL) was stirred at RT under H₂. The reaction was monitored by TLC until the reaction was complete. The Pd(OH)₂ was removed by filtration. The organic phase was concentrated under reduced pressure to afford a crude residue which was purified by flash chromatography (eluted with DCM/MeOH=50:1) to afford the title compound (30 mg, 54%) as an oil. LCMS: m/z 189 [M+H]⁺.

Step 5: Synthesis of N-(3-chloro-5-methylbenzyl)-1-(1-methyl-1H-indol-3-yl) propan-2-amine (6). The same procedure used for the synthesis of 5 was applied to afford the title compound. LCMS: m/z 328.10 [M+H]⁺. ¹H NMR (400 MHz, CDCl₃) δ 7.55 (d, J=8.0 Hz, 1H), 7.29 (d, J=8.2 Hz, 1H), 7.23 (d, J=7.2 Hz, 1H), 7.13-7.05 (m, 1H), 6.98 (d, J=4.9 Hz, 2H), 6.83 (d, J=7.7 Hz, 2H), 3.80 (d, J=13.7 Hz, 1H), 3.75 (s, 3H), 3.65 (d, J=13.7 Hz, 1H), 3.03 (d, J=6.5 Hz, 1H), 2.84 (dd, J=6.6, 3.4 Hz, 2H), 2.22 (s, 3H), 1.15 (d, J=6.2 Hz, 3H).

### Example 7: Synthesis of N-(3-chloro-5-methylbenzyl)-2-(1-ethyl-1H-indol-3-yl) ethan-1-amine hydrochloride (7)

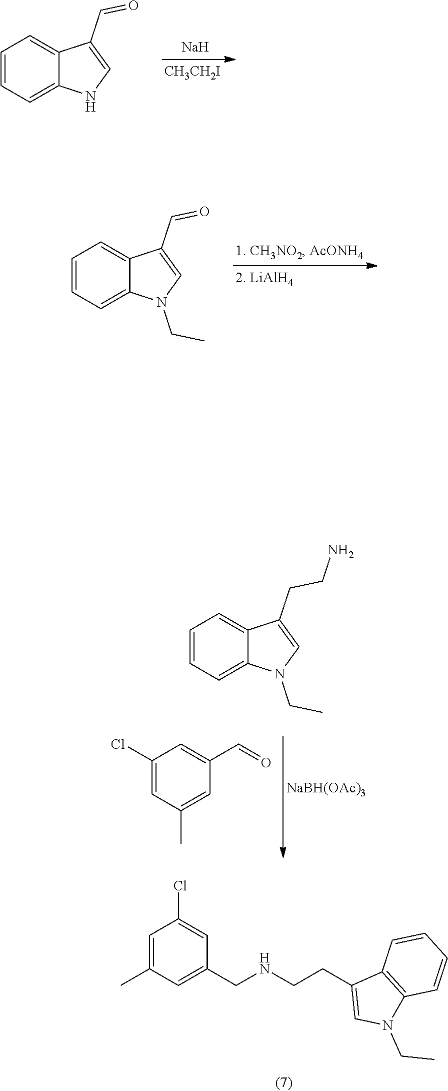

Step 1: Synthesis of 1-ethyl-1H-indole-3-carbaldehyde. A suspension of 1H-indole-3-carbaldehyde (2 g, 13.7 mmol, 1.0 eq) and NaH (0.98 g, 41.1 mmol, 3.0 eq) in DMF (15 mL) was stirred at RT for 30 min under N₂. CH₃CH₂I (3.2 g, 20.6 mmol, 1.5 eq) was added slowly and the resulting mixture was stirred at RT overnight. Water (200 mL) was added to quench the reaction and the mixture was extracted with EA (50 mL×3). The combined organic extracts were dried over anhydrous sodium sulfate and concentrated under reduced pressure to give a crude residue which was purified by flash chromatography (eluted with petroleum ether/EA=5:1) to afford the title compound as an oil (2.17 g, 87%).

Step 2: Synthesis of (E)-1-ethyl-3-(2-nitrovinyl)-1H-indole. A suspension of 1-ethyl-1H-indole-3-carbaldehyde (0.5, 3 mmol, 1.0 eq), nitromethane (1 g, 15.0 mmol, 5.0 eq) and ammonium acetate (0.4 g, 15.0 mmol, 5.0 eq) in 10 mL acetic acid was heated to 60° C. overnight. A yellow solid formed. The reaction was cooled to RT, 40 mL water and 3 mL EtOH were added and the resulting mixture was stirred 30 min. The reaction was washed with water (10 mL×3) and dried at 50° C. to afford the title compound 0.71 g (87%) as a yellow solid.

Step 3: Synthesis of 2-(1-ethyl-1H-indol-3-yl) ethan-1-amine. A suspension of LiBH₄ (64 mg, 2.97 mmol, 4.0 eq) in 15 mL THF was cooled to 0° C. under N₂. TMSCl (641 g, 5.93 mmol, 8.0 eq) was added slowly at 0° C. After the addition was complete, the resulting mixture was stirred for 30 min at 0° C. (E)-1-ethyl-3-(2-nitrovinyl)-1H-indole (150 mg, 0.74 mmol, 1.0 eq) dissolved in 5 mL THF was added to the reaction and the resulting mixture was heated at reflux for 4 h. The reaction was monitored by TLC till the reaction was complete. Water (20 mL) was added to quench the reaction and Na₂CO₃ (aq.) was added to adjust pH to 11. The resulting mixture was then extracted with DCM (15 mL×3). The combined organic extracts were dried over Na₂SO₄ and concentrated under reduced pressure to afford a crude residue which was purified by flash chromatography (Eluted with DCM/CH₃OH=20:1) to afford the title compound (80 mg, 62%) as an oil. LCMS: m/z 189 [M+H]⁺.

Step 4: Synthesis of N-(3-chloro-5-methylbenzyl)-2-(1-ethyl-1H-indol-3-yl) ethan-1-amine (7). The same procedure as was used to prepare 1 was applied to afford the title compound which was converted to the HCl salt (40 mg, 29%) as a white solid after treatment with HCl in dioxane. LCMS: m/z 327.10 [M+H]⁺. ¹H NMR (400 MHz, MeOD) δ 7.57 (d, J=7.9 Hz, 1H), 7.42 (d, J=8.3 Hz, 1H), 7.34 (d, J=7.1 Hz, 2H), 7.28-7.16 (m, 3H), 7.13-7.04 (m, 1H), 4.86 (s, 5H), 4.27-4.16 (m, 4H), 3.36 (dd, J=6.5, 4.5 Hz, 2H), 3.24-3.15 (m, 2H), 2.39 (d, J=0.4 Hz, 3H), 1.44 (t, J=7.2 Hz, 3H).

### Example 8: Synthesis of N-(3-chloro-5-methylbenzyl)-1-(1-ethyl-1H-indol-3-yl) propan-2-amine (8)

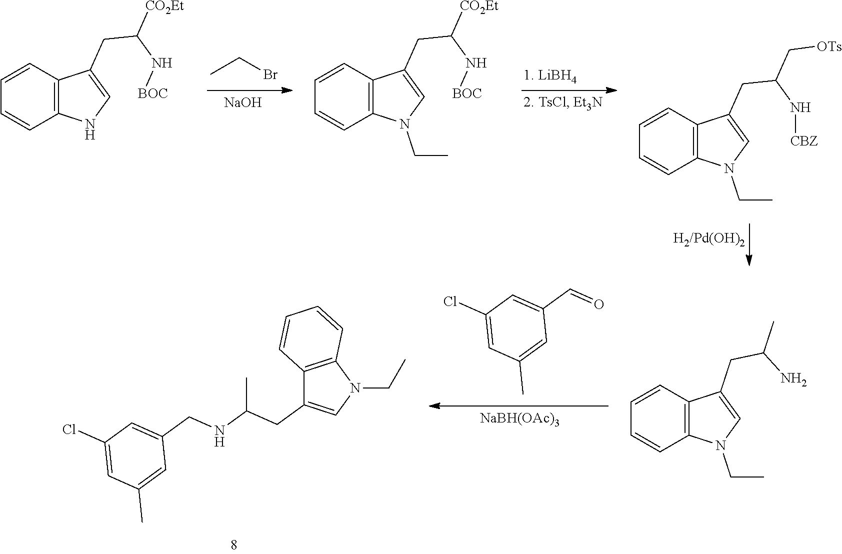

Step 1: Synthesis of Ethyl N-(tert-butoxycarbonyl)-1-ethyltryptophanate. A suspension of ethyl (tert-butoxycarbonyl) tryptophanate (5 g, 16 mmol, 1.0 eq) and NaOH (1.3 g, 32 mmol, 2.0 eq) in DMSO (20 mL) was heat ed to 40° C. overnight. Water (250 mL) was added and the resulting mixture was extracted with EA (100 mL×3). The combined organic extracts were dried over anhydrous sodium sulfate and concentrated under reduced pressure to give a crude residue which was purified by flash chromatography (eluted with petroleum ether/EA=10:1 gradient) to afford the title compound (3.6 g oil, 62%). LCMS: m/z 361.3. ¹H NMR (400 MHz, CDCl₃) δ 7.55 (d, J=7.9 Hz, 1H), 7.30 (d, J=8.2 Hz, 1H), 7.23-7.16 (m, 1H), 7.09 (t, J=7.5 Hz, 1H), 6.92 (s, 1H), 4.17-4.07 (m, 5H), 1.42 (t, J=7.3 Hz, 11H), 1.25 (dd, J=9.4, 4.9 Hz, 1H), 1.19 (t, J=7.1 Hz, 3H).

Step 2: Synthesis of tert-butyl (1-(1-ethyl-1H-indol-3-yl)-3-hydroxypropan-2-yl)carbamate. A suspension of LiBH₄ (0.99 g, 45 mmol, 5.0 eq) in 30 mL THF was cooled to 0° C. under N₂, ethyl N-(tert-butoxycarbonyl)-1-ethyltryptophanate (3.3 g, 9 mmol, 1.0 eq) dissolved in 10 mL THF was added to the above solution at 0° C., and the resulting mixture was stirred at RT overnight. CH₃OH (50 mL) was added to quench the reaction. The resulting mixture was concentrated under reduced pressure to give a crude residue which was purified by flash chromatography (eluted with DCM/CH₃OH=50:1) to afford the title compound (2.7 g oil, 93%). LCMS: m/z 319. ¹H NMR (400 MHz, CDCl₃) δ 7.63 (d, J=7.9 Hz, 1H), 7.32 (d, J=8.2 Hz, 1H), 7.21 (d, J=1.0 Hz, 1H), 7.14-7.07 (m, 1H), 6.98 (s, 1H), 5.29 (s, 1H), 4.87-4.73 (m, 1H), 4.13 (q, J=7.3 Hz, 2H), 3.97 (dd, J=10.3, 5.9 Hz, 1H), 3.76-3.53 (m, 2H), 2.98 (d, J=6.8 Hz, 2H), 1.64 (s, 2H), 1.47-1.38 (m, 12H).

Step 3: Synthesis of 2-amino-3-(1-ethyl-1H-indol-3-yl) propyl 4-methylbenzenesulfonate. A suspension of tert-butyl (1-(1-ethyl-1H-indol-3-yl)-3-hydroxypropan-2-yl)carbamate (250 mg, 0.78 mmol, 1.0 eq) and TEA (158 mg, 1.57 mmol, 2.0 eq) in 10 mL DCM was cooled to 0° C. TsCl (158 mg, 0.83 mmol, 1.06 eq) was added to the solution at 0° C. and the reaction was stirred at RT overnight. The reaction was concentrated under reduced pressure to give a crude residue which was purified by flash chromatography (eluted with DCM/CH₃OH=20:1) to afford the title compound as an oil (90 mg, 30%).

Step 4: Synthesis of 1-(1-ethyl-1H-indol-3-yl) propan-2-amine. A suspension of 2-amino-3-(1-ethyl-1H-indol-3-yl) propyl 4-methylbenzenesulfonate (90 mg, 0.24 mmol, 1.0 eq) and Pd(OH)₂ (9 mg, 10% w/w) in 10 mL EtOH was stirred at RT under H₂. When the reaction was complete the Pd(OH)₂ was filtered. And organic layer was concentrated under reduced pressure to give the title compound as an oil (50 mg, 99%).

Step 5: Synthesis of N-(3-chloro-5-methylbenzyl)-1-(1-ethyl-1H-indol-3-yl) propan-2-amine (8). The same procedure used for the synthesis of 5 was applied to afford the title compound (40 mg, 47%) as an oil. LCMS: m/z 342.85 [M+H]⁺. ¹H NMR (400 MHz, CDCl₃) δ 7.57 (d, J=7.9 Hz, 1H), 7.33 (d, J=8.2 Hz, 1H), 7.22 (s, 1H), 7.13-7.06 (m, 1H), 7.00 (s, 2H), 6.94 (s, 1H), 6.84 (s, 1H), 4.14 (q, J=7.3 Hz, 2H), 3.81 (d, J=13.6 Hz, 1H), 3.67 (d, J=13.6 Hz, 1H), 3.05 (d, J=6.5 Hz, 1H), 2.86 (dd, J=6.6, 2.3 Hz, 2H), 2.23 (s, 3H), 1.45 (t, J=7.3 Hz, 3H), 1.17 (d, J=6.2 Hz, 3H).

### Example 9: Synthesis of N-(3-chloro-5-methylbenzyl)-2-(2-methyl-1H-indol-3-yl) ethan-1-amine hydrochloride (9)

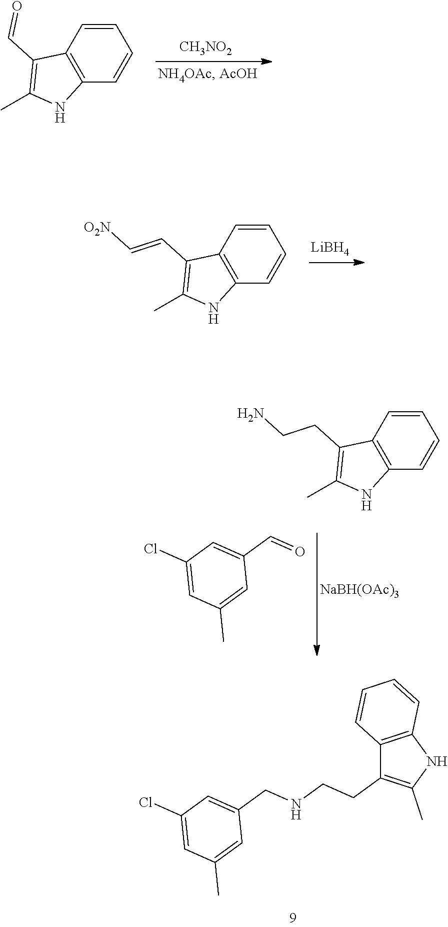

Step 1: Synthesis of (E)-2-methyl-3-(2-nitrovinyl)-1H-indole. A suspension of 2-methyl-1H-indole-3-carbaldehyde (0.5, 3.1 mmol, 1.0 eq), nitromethane (1 g, 15.0 mmol, 5.0 eq) and ammonium acetate (0.4 g, 15.0 mmol, 5.0 eq) in 10 mL acetic acid was heated to 60° C. overnight. The reaction was cooled to RT and water (40 mL) and EtOH (3 mL) were added. The resulting mixture was stirred 30 min at RT. The reaction mixture was filtered, washed with water (10 mL×3) and dried at 50° C. to afford the title compound (0.22 g, 36%) as a yellow solid.

Step 2: Synthesis of 2-(2-methyl-1H-indol-3-yl) ethan-1-amine. A suspension of LiBH₄ (86 mg, 3.96 mmol, 4.0 eq) in 15 mL THF was cooled to 0° C. under N₂. TMSCl (855 mg, 7.92 mmol, 8.0 eq) was added to the solution slowly at 0° C. After the addition was complete the resulting mixture was stirred for 30 min at 0° C. (E)-2-methyl-3-(2-nitrovinyl)-1H-indole (200 mg, 0.99 mmol, 1.0 eq) dissolved in 5 mL THF was added to the mixture and the reaction was heated to reflux for 4 h. The reaction was monitored by TLC until the reaction was complete. Water (20 mL) was added to quench the reaction and Na₂CO₃ (aq.) was added to adjust pH to 11. The resulting mixture was then extracted with DCM (15 mL×3). The combined organic extracts were dried over Na₂SO₄ and concentrated under reduced pressure to afford a crude residue which was purified by flash chromatography (Eluted with DCM/CH₃OH=20:1) to afford the title compound (120 mg, 70%) as an oil. LC_MS: m/z 349 [M+H]⁺.

Step 3: Synthesis of N-(3-chloro-5-methylbenzyl)-2-(2-methyl-1H-indol-3-yl) ethan-1-amine (9). The same procedure used for the preparation of 5 was applied to afford the title compound which was converted to the HCl salt (45 mg, 23%) as a white solid after treatment with HCl in dioxane. LCMS: m/z 313 [M+H]⁺. ¹H NMR (400 MHz, CDCl₃) δ 9.66-9.29 (m, 2H), 7.89 (s, 1H), 7.33 (d, J=7.7 Hz, 1H), 7.28 (s, 1H), 7.16-7.04 (m, 3H), 7.00 (s, 1H), 6.94 (s, 1H), 4.93 (s, 1H), 3.92 (s, 2H), 3.18-3.01 (m, 2H), 2.95-2.82 (m, 2H), 2.32 (s, 3H), 2.14 (s, 6H).

### Example 10: Synthesis of N-(3-fluoro-5-methylbenzyl)-2-(1H-indol-3-yl) ethan-1-amine hydrochloride (10)

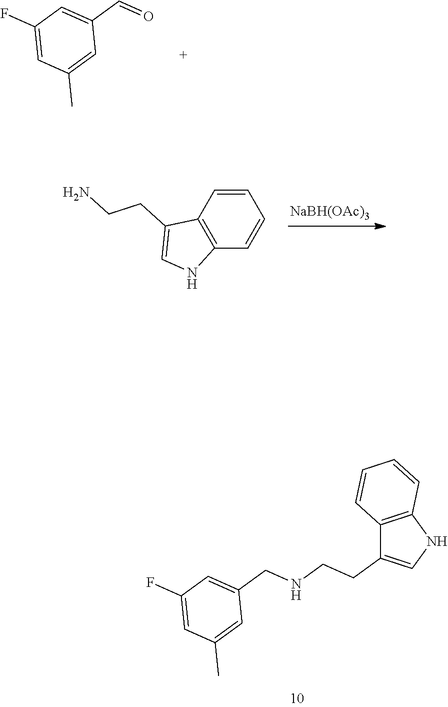

A solution of 3-fluoro-5-methylbenzaldehyde (42.5 mg, 0.31 mmol, 1 eq), 2-(1H-indol-3-yl) ethan-1-amine (50 mg, 0.31 mmol, 1 eq) and NaBH(OAc)₃ (200 mg, 0.93 mmol, 3 eq) in 3 mL DCM was stirred at RT for 16 h. The reaction was washed by NaHCO₃ (aq.) and extracted with DCM. The combined organic extracts were collected and concentrated under reduced pressure. The resulting residue was purified by flash chromatography (eluting with DCM/MeOH=20/1) to afford the title compound which was treated with 4M HCl in dioxane as the HCl salt (50 mg, 51% yield). LCMS: 1.073 min, m/z 283.45 [M+H]⁺. ¹H NMR (400 MHz, MeOD) δ 7.55 (d, J=7.9 Hz, 1H), 7.38 (d, J=8.1 Hz, 1H), 7.18 (s, 1H), 7.16-7.09 (m, 2H), 7.04 (t, J=7.6 Hz, 3H), 4.19 (s, 2H), 3.38-3.32 (m, 2H), 3.24-3.15 (m, 2H), 2.38 (s, 3H).

### Example 11: Synthesis of N-(3-chloro-5-methylbenzyl)-2-(2H-indazol-2-yl) ethan-1-amine (11)

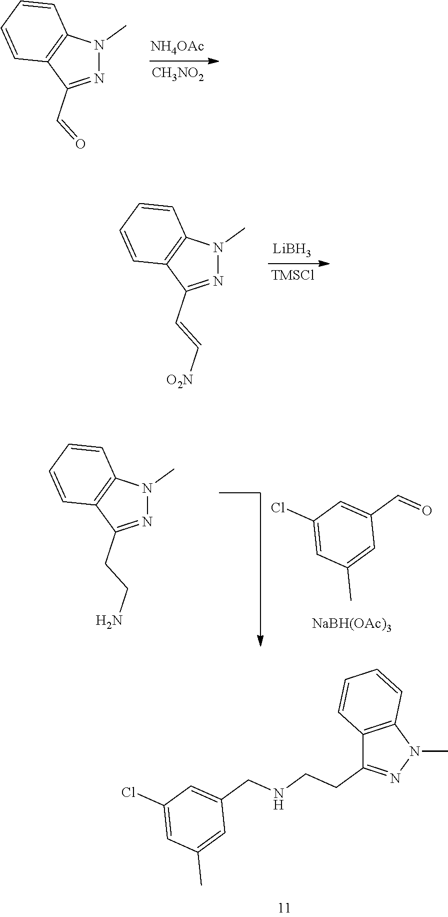

Step 1: Synthesis of (E)-1-methyl-3-(2-nitrovinyl)-1H-indazole. A suspension of 1-methyl-1H-indazole-3-carbaldehyde (800 mg, 4.9 mmol, 1.0 eq), nitromethane (2 ml) and ammonium acetate (1.15 g, 14.9 mmol, 3.0 eq) in acetic acid (6 mL) was heated to 60° C. overnight. The reaction was monitored by TLC until the reaction was complete. The reaction was cooled to RT, water (40) and EtOH (3 mL) were added. The resulting mixture was stirred 30 min at RT, filtered, washed with water (10 mL×3) and dried at 50° C. to afford the title compound (0.70 g, 69%) as a yellow solid.

Step 2: Synthesis of 2-(1-methyl-1H-indazol-3-yl) ethan-1-amine. A suspension of LiBH₄ (125 mg, 5.76 mmol, 4.0 eq) in THF (15 mL) was cooled to 0° C. under N₂. TMSCl (1.24 g, 11.52 mmol, 8.0 eq) was added slowly at 0° C. After the addition was complete, the resulting mixture was stirred for 30 min at 0° C. (E)-1-methyl-3-(2-nitrovinyl)-1H-indazole (300 mg, 1.44 mmol, 1.0 eq) dissolved in 5 mL THF was added to the mixture and the resulting mixture was heated to reflux for 4 h. The reaction was monitored by TLC until the reaction was complete. Water (20 mL) was added to quench the reaction and Na₂CO₃ (aq.) was added to adjust the pH to 11. The resulting mixture was extracted with DCM (15 mL×3). The combined organic phases were dried over Na₂SO₄ and concentrated under reduced pressure to afford a crude residue which was purified by flash chromatography (eluted with DCM/CH₃OH=10:1) to afford the title compound (90 mg, 36%) as an oil. LCMS: m/z 176 [M+H]⁺.

Step 3: Synthesis of N-(3-chloro-5-methylbenzyl)-2-(1-methyl-1H-indazol-3-yl)-ethan-1-amine. A solution of 2-(1-methyl-1H-indazol-3-yl) ethan-1-amine (50 mg, 0.28 mmol, 1 eq) and 3-chloro-5-methylbenzaldehyde (43.9 mg, 0.28 mmol, 1 eq) in DCM (3 mL) was stirred at RT for 1 h, followed by addition of NaBH(OAc)₃ (180 mg, 0.85 mmol, 3 eq). The resulting mixture was stirred at RT for 16 h. The reaction mixture was washed with NaHCO₃ (aq.) and extracted with DCM. The organic phase was concentrated under reduced pressure to afford a residue which was purified by flash chromatography (DCM/MeOH=30/1) to obtain crude product which was purified by preparative-HPLC to afford the title compound (40 mg, 33%) as a solid. LCMS: m/z 314.25 [M+H]⁺. ¹H NMR (400 MHz, CDCl₃) 10.30-9.98 (m, 1H), 7.56 (d, J=8.2 Hz, 1H), 7.37 (s, 1H), 7.33 (s, 1H), 7.25 (s, 1H), 7.16 (s, 1H), 7.13 (dd, J=4.7, 2.0 Hz, 2H), 4.13 (s, 2H), 3.99 (s, 3H), 3.45 (s, 2H), 3.30 (s, 2H), 2.30 (s, 3H).

### Example 12: Synthesis of N-(3-chloro-5-methylbenzyl)-2-(1H-indazol-1-yl) ethan-1-amine hydrochloride (12)

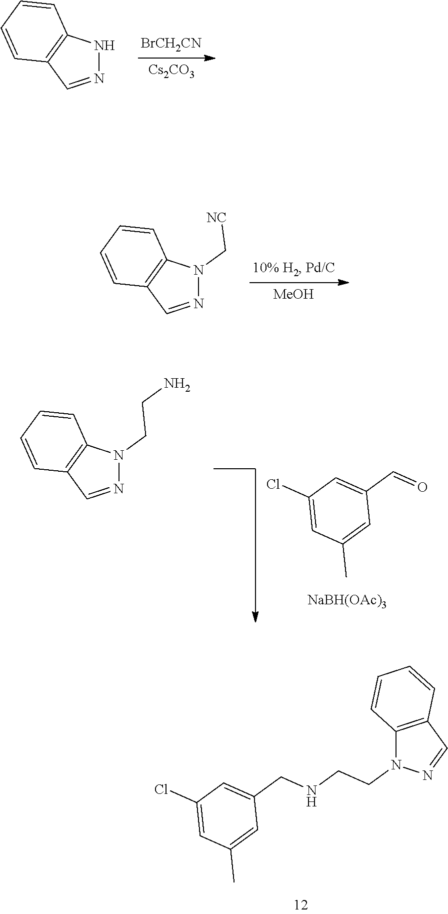

Step 1: Synthesise of 2-(1H-indazol-1-yl) acetonitrile. A solution of 1H-indazole (1 g, 8.5 mmol, 1.0 eq), Cs₂CO₃ (5.52 g, 17 mmol, 2.0 eq) and 2-bromoacetonitrile (2 g, 17 mmol, 2.0 eq) in DMF (30 mL) was stirred at RT for 16 h. The reaction was poured into water and the resulting mixture was extracted with DCM (50 mL×3). The combined organic extracts were concentrated to afford a residue which was purified by silica gel chromatography (DCM/MeOH=20/1) to afford the title compound as a solid (500 mg, 37%).

Step 2: Synthesis of 2-(1H-indazol-1-yl) ethan-1-amine. A solution of 2-(1H-indazol-1-yl) acetonitrile (500 mg, 3.18 mmol) and 10 wt % Pd/C (30 mg) in MeOH (10 mL) was stirred under H₂ at RT for 16 h. The reaction mixture was filtered, the organic phase was collected and concentrated under reduced pressure to afford the title compound as a colorless oil (100 mg, 23%).

Step 3: Synthesis of N-(3-chloro-5-methylbenzyl)-2-(1H-indazol-1-yl) ethan-1-amine hydrochloride. A solution of 2-(1H-indazol-1-yl) ethan-1-amine (100 mg, 0.62 mmol, 1 eq) and 3-chloro-5-methylbenzaldehyde (96 mg, 0.62 mmol, 1 eq) in DCM (10 mL) was stirred at RT for 1 h followed by the addition of NaBH(OAc)₃ (477 mg, 2.25 mmol, 3 eq). The resulting mixture was stirred at RT for 16 h. The reaction was washed with NaHCO₃ (aq.) and extracted with DCM. The organic phase was concentrated under reduced pressure to afford a residue which was purified by flash chromatography (DCM/MeOH=10/1) to afford a crude product which was treated with 4 M HCl in dioxane to afford the title compound as a solid (67 mg, 30%). LCMS: (ES+): m/z 300.25 [M+H]. ¹H NMR (400 MHz, MeOD) δ 8.15 (d, J=0.8 Hz, 1H), 7.82 (d, J=8.2 Hz, 1H), 7.64 (dd, J=8.5, 0.7 Hz, 1H), 7.52-7.47 (m, 1H), 7.36 (s, 1H), 7.33 (s, 1H), 7.27-7.21 (m, 2H), 4.79 (t, J=5.9 Hz, 2H), 4.26 (s, 2H), 3.64 (t, J=5.9 Hz, 2H), 2.38 (s, 3H).

### Example 13: Synthesis of 2-(1H-benzo[d]imidazol-1-yl)-N-(3-chloro-5-methylbenzyl) ethan-1-amine hydrochloride (13)

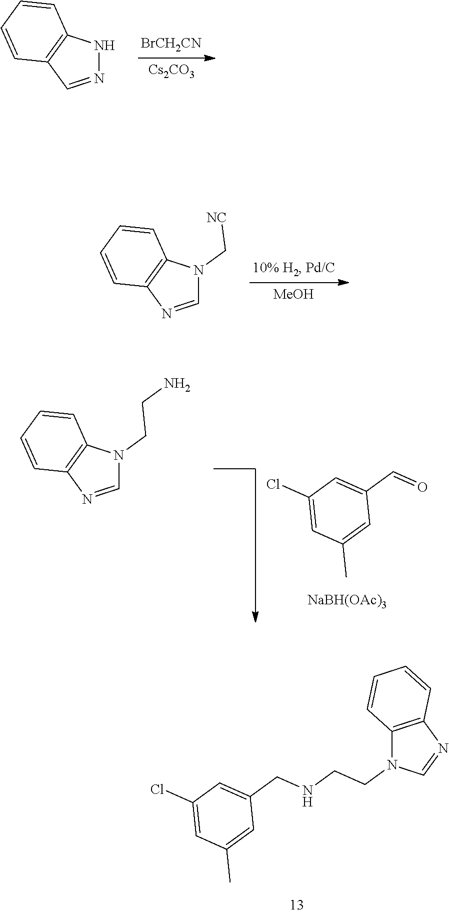

Step 1: Synthesis of 2-(1H-benzo[d]imidazol-1-yl) acetonitrile. A solution of 1H-benzo[d]imidazole (1 g, 8.5 mmol, 1 eq), Cs₂CO₃ (5.52 g, 17 mmol, 2.0 eq) and 2-bromoacetonitrile (2 g, 17 mmol, 2.0 eq) in DMF (30 mL) was stirred at RT for 16 h. The reaction was poured into water and the resulting mixture was extracted by DCM (50 mL×3). The combined organic extracts were concentrated to afford a residue which was purified by silica gel chromatography (DCM/MeOH=20/1) to afford the title compound as a solid (600 mg, 42%).

Step 2: Synthesis of 2-(1H-benzo[d]imidazol-1-yl) ethan-1-amine. A solution of 2-(1H-benzo[d]imidazol-1-yl) acetonitrile (600 mg, 3.18 mmol, 1 eq) and 10 w1% Pd/C (30 mg) in MeOH (10 mL) was stirred under H₂ at RT for 16 h. The reaction was filtered, the organic phase was collected and concentrated under reduced pressure to afford the title compound as a colorless oil (100 mg, 23%).

Step 3: Synthesis of 2-(1H-benzo[d]imidazol-1-yl)-N-(3-chloro-5-methylbenzyl) ethan-1-amine hydrochloride. A solution of 2-(1H-benzo[d]imidazol-1-yl) ethan-1-amine (100 mg, 0.62 mmol, 1 eq) and 3-chloro-5-methylbenzaldehyde (95.5 mg, 0.62 mmol, 1 eq) in DCM (10 mL) was stirred at RT for 1 h followed by the addition of NaBH(OAc)₃ (477 mg, 2.25 mmol, 3 eq). The resulting mixture was stirred at RT for 16 h.

The reaction was washed by NaHCO₃ (aq.), extracted by DCM. The organic phase was concentrated under reduced pressure to afford a residue which was purified by flash chromatography (DCM/MeOH=10/1) to afford the target which was treated with 4 M HCl in dioxane to afford the title compound as a solid (15 mg, 8%). LCMS: (ES+): m/z 300.10 [M+H]⁺. ¹H NMR (400 MHz, MeOD) δ 9.56 (s, 1H), 8.08 (d, J=7.6 Hz, 1H), 7.90 (d, J=7.7 Hz, 1H), 7.78-7.65 (m, 2H), 7.41 (s, 1H), 7.34 (s, 1H), 7.29 (s, 1H), 5.02 (s, 2H), 4.27 (s, 2H), 3.78 (d, J=5.5 Hz, 2H), 2.36 (s, 3H).

### Example 14: Synthesis of N-(3-chloro-5-methylbenzyl)-2-(1H-indazol-3-yl) ethan-1-amine (14)

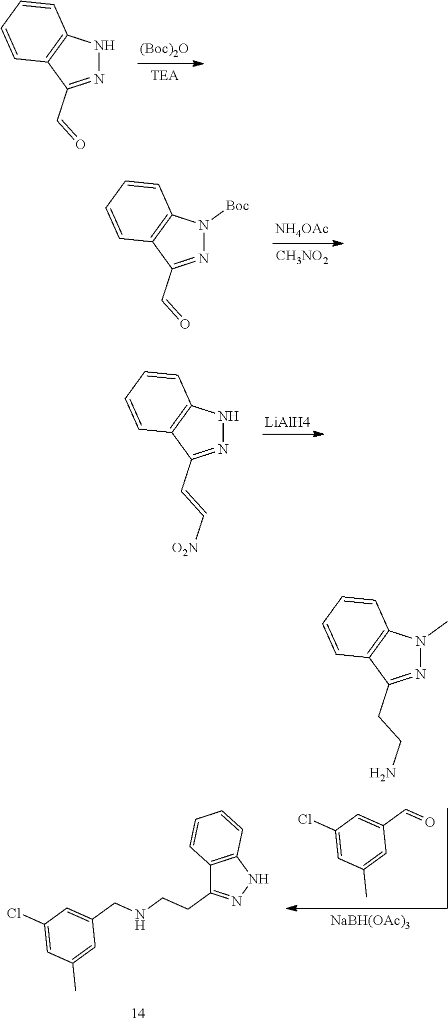

Step 1: Synthesis of tert-butyl 3-formyl-1H-indazole-1-carboxylate. A suspension of 1H-indazole-3-carbaldehyde (5 g, 34 mmol, 1 eq), (Boc) 20 (8.9 g, 41 mmol, 1.2 eq), TEA (4.14 g, 41 mmol, 1.2 eq) and DMAP (41 mg, 0.34 mmol, 0.01 eq) in DCM (80 mL) was stirred at RT overnight. Solvent was removed under reduced pressure to afford a crude residue which was purified by flash chromatography (eluted with Petroleum ether/EA=30:1) 10 to afford the title compound (4 g, 48% yield) as an oil.

Step 2: Synthesis of (E)-3-(2-nitrovinyl)-1H-indazole. A suspension of tert-butyl 3-formyl-1H-indazole-1-carboxylate (1 g, 12.5 mmol, 1.0 eq.), nitromethane (3 ml) and ammonium acetate (3 g, 38 mmol, 3.0 eq.) in 5 mL acetic acid was heated to 30° C. for 3 h and then heated to 60° C. overnight. The reaction was monitored by TLC until the reaction was complete and a yellow solid appeared. The reaction mixture was cooled to RT. Water (40 mL) and EtOH (3Ml) were added, and the resulting mixture was stirred 30 min at RT. The mixture was filtered, washed with water (10 mL×3) and dried at 50° C. to afford the title compound (600 mg, 79%) as a yellow solid.

Step 3 Synthesis of 2-(1H-indazol-3-yl) ethan-1-amine. A suspension of LiAlH₄ (479 mg, 12.7 mmol, 4.0 eq) in 15 mL THF was cooled to 0° C. under N₂. (E)-3-(2-nitrovinyl)-1H-indazole (600 mg, 3.17 mmol, 1.0 eq) dissolved in 5 mL THF was added to the mixture, and the resulting mixture was heated to reflux for 4 h. The reaction was monitored by TLC till the reaction was complete. Water (20 mL) was added to quench the reaction. The resulting mixture was then extracted with DCM (15 mL×3). The combined organic phase was died over Na₂SO₄ and concentrated under reduced pressure to afford a crude residue which was purified by flash chromatography (eluted with DCM/CH₃OH=10:1) to afford the title compound (305 mg, 59%) as an oil. LCMS: m/z 162 [M+H]⁺.

Step 4: Synthesis of N-(3-chloro-5-methylbenzyl)-2-(1H-indazol-3-yl) ethan-1-amine. A solution of 2-(1H-indazol-3-yl) ethan-1-amine (100 mg, 0.62 mmol, 1 eq) and 3-chloro-5-methylbenzaldehyde (95.6 mg, 0.62 mmol, 1 eq) in DCM (5 mL) was stirred at RT for 1 h followed by the addition of NaBH(OAc)₃ (394 mg, 1.86 mmol, 3 eq.). The resulting mixture was stirred at RT for 16 h. The reaction was washed by NaHCO₃ (aq.) and extracted with DCM. The organic phase was concentrated under reduced pressure to afford a residue which was purified by flash chromatography (DCM/MeOH=25/1) to afford the title compound as a solid. (70 mg, 37%) LCMS: m/z 300.20 [M+H]⁺. ¹H NMR (400 MHz, CDCl₃) δ 7.70 (d, J=8.1 Hz, 1H), 7.43 (s, 1H), 7.38 (s, 1H), 7.15 (s, 1H), 7.10 (s, 1H), 7.02 (s, 1H), 6.98 (s, 1H), 3.79 (s, 2H), 3.21 (d, J=6.3 Hz, 2H), 3.12 (d, J=6.3 Hz, 2H), 2.28 (s, 3H).

### Example 15: Synthesis of N-(3-chloro-5-methylbenzyl)-2-(imidazo[1,5-a]pyridin-3-yl)-ethan-1-amine (15)

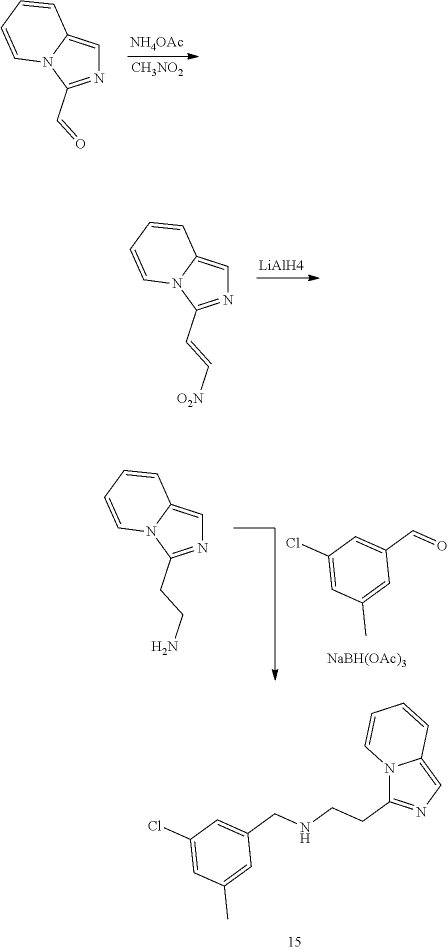

Step 1: Synthesis of 3-(2-nitrovinyl) imidazo[1,5-a]pyridine. A suspension of imidazo[1,5-a]pyridine-3-carbaldehyde (1 g, 6.8 mmol, 1 eq) and ammonium acetate (1.32 g, 17 mmol, 2.5 eq) in nitromethane (10 mL) was stirred at 90° C. for 1 h. and a yellow solid formed. Water (5 mL) and EtOH (5 mL) were added, and the mixture was stirred for 30 min at RT. The yellow solid was filtered, washed by water (10 mL×3) and dried at 50° C. to afford the title compound as a yellow solid (672 mg 51%).

Step 2: Synthesis of 2-(imidazo[1,5-a]pyridin-3-yl) ethan-1-amine. A suspension of LAH (675 mg, 18 mmol, 5 eq) in 5 mL THF was cooled to 0° C. under nitrogen and a solution (E)-3-(2-nitrovinyl) imidazo[1,5-a]pyridine (672 mg, 3.6 mmol, 1 eq) in THF (5 mL) was added. The resulting mixture was heated to reflux. When the reaction was complete, water (2 mL) was added to quench LAH followed Na₂CO₃ to adjust the pH to 11. The resulting mixture was filtered and the filtrate was concentrated under reduced pressure to afford the crude product which was purified by flash chromatography (eluted with DCM/CH₅OH=20:1) to afford the title compound (190 mg, 33%) as a colorless oil. LCMS: (ES+): 163.00 m/z [M+H]⁺.

Step 3: Synthesis of N-(3-chloro-5-methylbenzyl)-2-(imidazo[1,5-a]pyridin-3-yl)-ethan-1-amine. To a stirred solution of 2-(imidazo[1,5-a]pyridin-3-yl) ethan-1-amine (50 mg, 0.3 mmol, 1.0 eq) and 3-chloro-5-methylbenzaldehyde (33 mg, 0.22 mmol, 0.7 eq) in MeOH (5 mL) was added NaBH₃CN (56 mg, 0.9 mmol, 3.0 eq). The resulting mixture was stirred at RT overnight. The reaction mixture was quenched with aqueous sodium bicarbonate solution and extracted with ethyl acetate. The organic layer was dried over anhydrous sodium sulfate and concentrated under reduced pressure to give a crude residue which was purified by flash chromatography (eluted with PE/EA=1:1) to afford the title compound which was converted to the HCl salt (68 mg, 90%) as a white solid after treatment with HCl in dioxane. LCMS: (ES+): 301.30 m/z [M+H]⁺. ¹H NMR (400 MHz, Methanol-d₄) δ 8.33-8.28 (m, 1H), 7.79 (d, J=0.8 Hz, 1H), 7.74 (dt, J=9.2, 1.2 Hz, 1H), 7.38 (d, J=1.7 Hz, 1H), 7.34 (s, 1H), 7.29 (s, 1H), 7.16-7.08 (m, 1H), 7.05 (td, J=6.9, 1.2 Hz, 1H), 4.29 (s, 2H), 3.72-3.65 (m, 2H), 3.65-3.58 (m, 2H), 2.39 (s, 3H).

### Example 16: Synthesis of N-(3-chloro-5-methylbenzyl)-2-(2H-indazol-2-yl) ethan-1-amine hydrochloride (16)

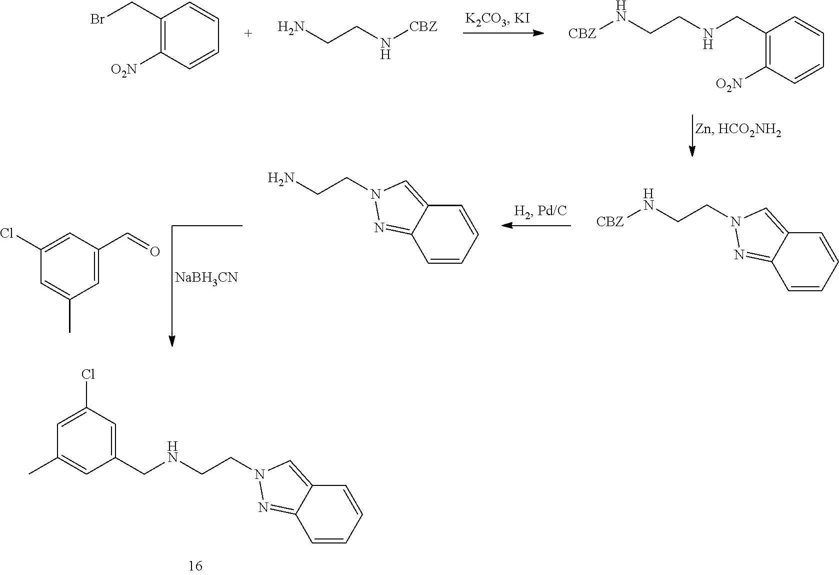

Step 1: Synthesis of Benzyl(2-((2-nitrobenzyl)amino)ethyl)carbamate. A solution of 1-(bromomethyl)-2-nitrobenzene (1.00 g, 4.63 mmol, 1 eq) in DMF (10 mL) was added benzyl(2-aminoethyl)-carbamate (1.09 g, 5.63 mmol, 1.2 eq), K₂CO₃ (958 mg, 6.95 mmol, 1.5 eq) and KI (230 mg, 1.39 mmol, 0.3 eq), and the reaction mixture was stirred at RT overnight. After the reaction was complete, water (100 mL) was added and the mixture was extracted with EtOAc (50 mL×3). The organic layer was washed with water (10 mL×3) and dried over Na₂SO₄ and concentrated under reduced pressure to give a crude residue which was purified by silica gel flash column chromatography (eluted with petroleum ether/EA=4:1) to afford the title compound (1.0 g, 65%) as a yellow oil. LCMS: (ES+): 330.65 m/z. [M+H]⁺.

Step 2: Synthesis of Benzyl(2-(2H-indazol-2-yl)ethyl)carbamate. A solution of benzyl(2-((2-nitrobenzyl)amino)ethyl)carbamate (1.0 g, 3.04 mmol, 1 eq) in MeOH (10 mL) was added Zn (1.0 g, 15.18 mmol, 5 eq) and HCOONH₄ (191.5 mg, 3.04 mmol, 1 eq) and the reaction mixture was stirred at RT overnight. After the reaction was complete, water (100 mL) was added and the mixture was extracted with EtOAc (50 mL×3). The organic layer was washed by water (10 mL×3), dried over Na₂SO₄ and concentrated under reduced pressure to give a crude residue which was purified by flash chromatography (eluted with petroleum ether/EA=5:1) to afford the title compound (330 mg, 37%) as a yellow oil. LCMS: (ES+): 296.70 m/z [M+H]⁺.

Step 3: Synthesis of 2-(2H-indazol-2-yl) ethan-1-amine. To a stirred solution of benzyl(2-(2H-indazol-2-yl)ethyl)carbamate (330 mg, 1.11 mmol, 1.0 eq) in MeOH (10 mL) was added Pd (90 mg). The resulting mixture was stirred at RT under N₂ overnight. The reaction mixture was filtered and the filtrate was concentrated under reduced pressure to afford the title compound (172 mg, 100%) which was used next step without further purification. LCMS: (ES+): 162.45 m/z [M+H]⁺.

Step 4: Synthesis of N-(3-chloro-5-methylbenzyl)-2-(2H-indazol-2-yl) ethan-1-amine (16). To a stirred solution of 2-(2,5-dimethoxypyridin-3-yl) ethan-1-amine (60 mg, 0.31 mmol, 1.0 eq) and 3-chloro-5-methy-1benzaldehyde (48 mg, 0.31 mmol, 1 eq) in DCM (5 mL) was added NaBH₃CN (58 mg, 1 mmol, 3.0 eq). The resulting mixture was stirred at RT overnight. The reaction mixture was quenched with aqueous sodium bicarbonate solution and extracted with ethyl acetate. The organic layer was dried over anhydrous sodium sulfate and concentrated under reduced pressure to give a crude residue which was purified by flash chromatography (eluted with petroleum ether/EA=1:1) to afford the title compound which was converted to the HCl salt (52 mg, 57%) as a white solid after treatment with HCl in dioxane. LCMS: (ES+): 301.40 m/z [M+H]⁺. t_{R}=2.157 min; ¹H NMR (400 MHz, CD₃OD) δ 8.34 (s, 1H), 7.73 (d, J=8.8 Hz, 1H), 7.66 (dd, J=8.8, 0.8 Hz, 1H), 7.37 (s, 1H), 7.36-7.33 (m, 1H), 7.30 (s, 1H), 7.26 (s, 1H), 7.15-7.11 (m, 1H), 4.87 (d, J=5.6 Hz, 2H), 4.26 (s, 2H), 3.67 (t, J=5.6 Hz, 2H), 2.35 (s, 3H).

### Example 17: Synthesis of 3-(2-((3-chloro-5-methylbenzyl)amino)ethyl)benzo-[d]oxazol-2(3H)-one (17)

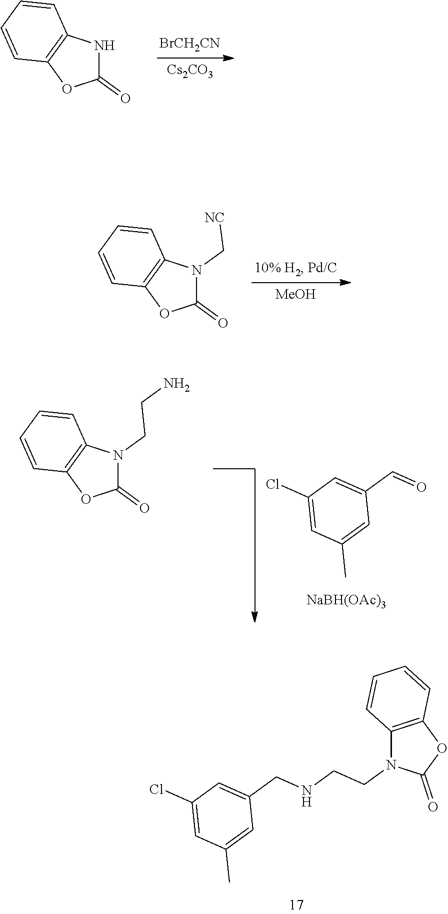

Step 1: Synthesis of 2-(2-oxobenzo[d]oxazol-3 (2H)-yl) acetonitrile. A solution of benzo[d]oxazol-2(3H)-one (3 g, 22.2 mmol, 1 eq), Cs₂CO₃ (5.32 g, 44 mmol, 2.0 eq) and 2-bromoacetonitrile (21.64 g, 66.6 mmol, 3.0 eq) in DMF (30 mL) was stirred at RT for 16 h. The reaction mixture was poured into water and the resulting mixture was extracted by DCM (50 mL×3). The combined organic phases were concentrated to afford a residue which was purified by chromatography (DCM/MeOH=20/1) to afford the title compound (400 mg, 10%) as a solid.

Step 2. Synthesis of 3-(2-aminoethyl)benzo[d]oxazol-2(3H)-one. A solution of 2-(2-oxobenzo[d]oxazol-3 (2H)-yl) acetonitrile (400 mg, 2.29 mmol, 1 eq) and 10 wt % Pd/C (40 mg) in MeOH (10 mL) was stirred under H₂ at RT for 16 h. The reaction mixture was filtered, the organic phase was collected and concentrated under reduced pressure to afford the title compound (140 mg, 34%) as a white solid.

Step 3: Synthesis of 3-(2-((3-chloro-5-methylbenzyl)amino)ethyl)benzo-└d┘oxazol-2(3H)-one. A solution of 3-(2-aminoethyl)benzo[d]oxazol-2(3H)-one (140 mg, 0.78 mmol, 1 eq) and 3-chloro-5-methylbenzaldehyde (121.5 mg, 0.78 mmol, 1 eq) in DCM (10 mL) was stirred at RT for 1 h followed by the addition of NaBH(OAc)₃ (499 mg, 2.25 mmol, 3 eq). The resulting mixture was stirred at RT for 16 h. The resulting mixture was washed with NaHCO₃ (aq.) and extracted with DCM. The organic phase was concentrated under reduced pressure to afford a residue which was purified by flash chromatography (Petroleum ether/EA=1/1) to afford the title compound (150 mg, 80%) as an oil. LCMS: (ES+): m/z 337.25 [M+H]⁺. ¹H NMR (400 MHz, CDCl₃) δ 7.21 (d, J=8.3 Hz, 1H), 7.16 (dd, J=7.6, 1.0 Hz, 1H), 7.12 (dd, J=7.7, 1.2 Hz, 1H), 7.05 (s, 1H), 7.02 (s, 1H), 6.99 (d, J=7.8 Hz, 1H), 6.93 (s, 1H), 3.94 (t, J=6.2 Hz, 2H), 3.75 (s, 2H), 3.00 (t, J=6.2 Hz, 2H), 2.27 (s, 3H).

### Example 18: Synthesis of Synthesis of N-(3-chloro-5-methylbenzyl)-2-(1H-indol-3-yl) ethan-1-amine (18)

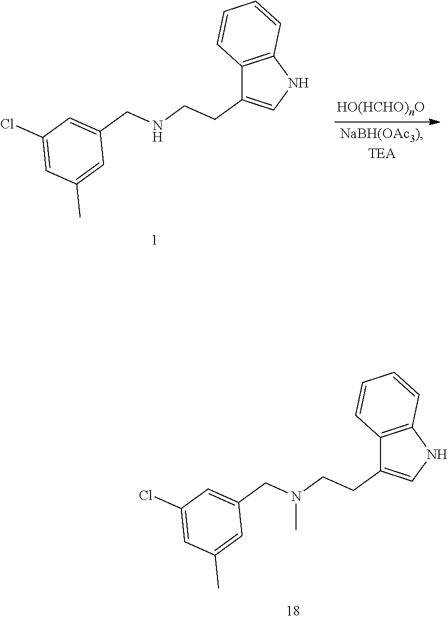 [SMILES: Cc1cc(Cl)cc(CNCCc2c[nH]c3ccccc23)c1 ; Cc1cc(Cl)cc(CN(C)CCc2c[nH]c3ccccc23)c1]

Synthesis of N-(3-chloro-5-methylbenzyl)-2-(1H-indol-3-yl) ethan-1-amine (18). A solution of N-(3-chloro-5-methylbenzyl)-2-(1H-indol-3-yl) ethan-1-amine hydrochloride, Compound 1 (500 mg, 1.49 mmol, 1 eq), paraformaldehyde (45 mg, 14.9 mmol, 10 eq), TEA (165 mg, 1.63 mmol, 1.1 eq) and NaBH(OAc)₃ (1.57 mg, 7.45 mmol, 5 eq) in DCE (10 mL) was heated to 50° C. and stirred overnight. The reaction mixture was washed with saturated NaHCO₃ (aq.) and extracted with DCM. The organic phase was concentrated under reduced pressure to afford a residue which was purified by flash chromatography (petroleum ether/EA=20/1) to afford the title compound (10 mg, 2%) as an oil. LCMS: (ES+): m/z 314.95 [M+H]⁺. ¹H NMR (400 MHz, CDCl₃) δ 7.97 (s, 1H), 7.56 (d, J=7.9 Hz, 1H), 7.34 (d, J=8.1 Hz, 1H), 7.18 (t, J=7.2 Hz, 1H), 7.13 (s, 1H), 7.10 (t, J=7.2 Hz, 1H), 7.04 (s, 1H), 7.00 (s, 2H), 3.51 (s, 2H), 3.02-2.95 (m, 2H), 2.77-2.69 (m, 2H), 2.32 (s, 3H), 2.29 (s, 3H).

### Example 19: Biological Studies

Biased signaling consequences of 5-HT2A activation by various agonists strongly impact whether a compound will be hallucinogenic or non-hallucinogenic. For example, LSD and lisuride both activate the 5-HT2A receptor but in slightly different ways which result in the activation of different intracellular signaling cascades. LSD and lisuride have been shown to activate canonical Gq-based signaling downstream of 5-HT2A, but only LSD stimulated the expression of early growth response proteins (EGR1 and EGR2) by activating Gi/o subunits and the SRC protein kinase (see, e.g., Gonzalez-Maeso et al, Neuron 53, 439-452 (2007). Differential functional selectivity has been shown for several phenalkylamine psychedelics which were found to be biased 5-HT2A agonists (see, e.g., Pottie et al, Biochemical pharmacology, 182, 114251, 2020). The compounds, including 25H-NBF, 25H-NBMD, 25H-NBOH and 25H-NBOMe showed a statistically significant preference towards the recruitment of β-arrestin 2 over miniGaq, as compared to the reference psychedelic substance LSD.

Differential biased agonism elicited across multiple classes of psychedelics may identify whether this functional selectivity can provide compounds with greater selectivity, fewer side effects, greater neuroplastic effects and improved therapeutic benefit. Such activity is shown for compounds described herein.

### GPCR Arrestin Assay Methods:

Compounds 1-18 were assessed for biological activity across a panel of 5-HT receptors including 5-HT2A, 5-HT2B, and 5-HT2C. Biased signaling was assessed by monitoring both intracellular Gq-mediated calcium release, as well as β-arrestin activation and recruitment to the GPCR. Table 2 is a summary of findings across these compounds and the 5-HT receptors tested.
(i) Arrestin Pathway Assay. The PathHunter® β-Arrestin assay monitors the activation of a GPCR in a homogenous, non-imaging assay format using a technology developed by DiscoverX called Enzyme Fragment Complementation (EFC) with β-galactosidase (β-Gal) as the functional reporter. The enzyme is split into two inactive complementary portions (EA for Enzyme Acceptor and PK for ProLink) expressed as fusion proteins in the cell. EA is fused to β-Arrestin and PK is fused to the GPCR of interest. When the GPCR is activated and β-Arrestin is recruited to the receptor, ED and EA complementation occurs, restoring β-Gal activity which is measured using chemiluminescent PathHunter® Detection Reagents. PathHunter cell lines (DiscoveRx Eurofins) were expanded from freezer stocks according to standard procedures. Cells were seeded in a total volume of 20 μL into white walled, 384-well microplates and incubated at 37° C. for the appropriate time prior to testing. For agonist determination, cells were incubated with sample to induce response. Intermediate dilution of sample stocks was performed to generate 5× sample in assay buffer. 5 μL of 5× sample was added to cells and incubated at 37° C. or room temperature for 90 to 180 minutes. Vehicle concentration was 1%. β-Arrestin assay signal was generated through a single addition of 12.5 or 15 μL (50% v/v) of PathHunter Detection reagent cocktail, followed by a one-hour incubation at room temperature. Microplates were read following signal generation with a PerkinElmer Envision™ instrument for chemiluminescent signal detection. Compound activity was analyzed using CBIS data analysis suite (ChemInnovation, CA). For agonist mode assays, percentage activity was calculated using the following formula: % Activity=100%×(mean RLU of test sample-mean RLU of vehicle control)/(mean MAX control ligand−mean RLU of vehicle control). In these studies, the MAX control ligand response was generated using 10 mM serotonin.
(ii) Calcium Mobilization Assay. The Calcium No-WashPLUS assay monitors the activation of a GPCR via Gq secondary messenger signaling in a live cell, non-imaging assay format. Calcium mobilization in PathHunter® cell lines or other cell lines stably expressing Gq-coupled GPCRs is monitored using a calcium-sensitive dye that is loaded into cells. GPCR activation by a compound result in the release of calcium from intracellular stores and an increase in dye fluorescence that is measured in real-time. Cell lines expressing the GPCR of interest were expanded from freezer stocks according to standard procedures. Cells were seeded in a total volume of 20 μL into black-walled, clear-bottom, Poly-D-lysine coated 384-well microplates and incubated at 37° C. for the appropriate time prior to testing. Assays were performed in 1× Dye Loading Buffer consisting of 1× Dye, 1× Additive A and 2.5 mM Probenecid in HBSS/20 mM Hepes. Probenicid was prepared fresh. Cells were loaded with dye prior to testing. Media was aspirated from cells and replaced with 20 μL Dye Loading Buffer. Cells were incubated for 30-60 minutes at 37° C. For agonist determination, cells were incubated with sample to induce response. After dye loading, cells were removed from the incubator and 10 μL HBSS/20 mM Hepes was added. 3× vehicle was included in the buffer when performing agonist dose curves to define the EC80 for subsequent antagonist assays. Cells were incubated for 30 minutes at room temperature in the dark to equilibrate plate temperature. Intermediate dilution of sample stocks was performed to generate 4× sample in assay buffer. Compound agonist activity was measured on a FLIPR Tetra (MDS). Calcium mobilization was monitored for 2 minutes and 10 μL 4× sample in HBSS/20 mM Hepes was added to the cells 5 seconds into the assay. Compound activity data was analyzed using CBIS data analysis suite (ChemInnovation, CA). For agonist mode assays, percentage activity is calculated using the following formula: % Activity=100%×(mean RFU of test sample−mean RFU of vehicle control)/(mean MAX RFU control ligand−mean RFU of vehicle control). In these studies, the MAX RFU was generated by using 0.1 mM serotonin for the calcium mobilization assay.

TABLE 2
Summary of Biological Data

| | | 5-HT2A, EC50 | | | |
|---|---|---|---|---|---|
| | | (□M)/ % activation | EC50 | | |
| | | @30 uM Ca⁺⁺ | 5-HT2A | EC50 | EC50 |
| | Structure | Flux | arrestin | 5-HT2C | 5-HT2B |
| 1 | 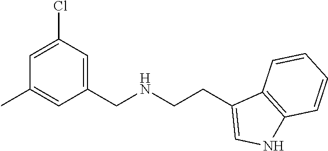 [SMILES: Cc1cc(Cl)cc(CNCCc2c[nH]c3ccccc23)c1] | 0.0024/77.8 | 0.0045 | 0.028 | >1 |
| 2 | 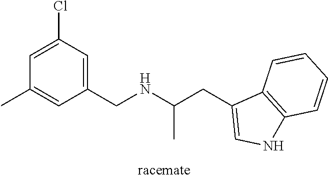 [SMILES: Cc1cc(Cl)cc(CNC(C)Cc2c[nH]c3ccccc23)c1] | 0.025/84.2 | 0.434 | 0.102 | >1 |
| 3 | 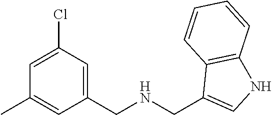 [SMILES: Cc1cc(Cl)cc(CNCc2c[nH]c3ccccc23)c1] | Not Tested/77.2 | Not Tested | Not Tested | Not Tested |
| 4 | 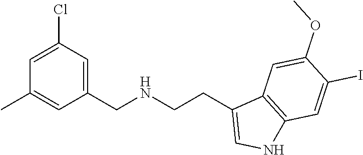 [SMILES: COc1cc2c(CCNCc3cc(C)cc(Cl)c3)c[nH]c2cc1I] | 0.254/96.4 | 0.060 | 0.384 | >1 |
| 5 | 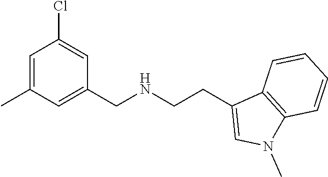 [SMILES: Cc1cc(Cl)cc(CNCCc2cn(C)c3ccccc23)c1] | 0.036/111.9 | 0.0175 | 0.116 | >1 |
| 6 | 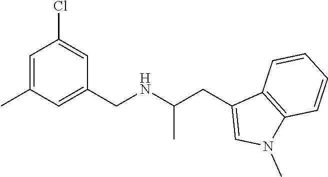 [SMILES: Cc1cc(Cl)cc(CNC(C)Cc2cn(C)c3ccccc23)c1] | Not Tested/109.9 | Not Tested | Not Tested | Not Tested |
| 7 | 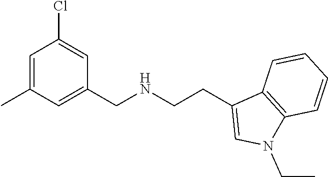 [SMILES: CCn1cc(CCNCc2cc(C)cc(Cl)c2)c2ccccc21] | 0.0546/95.9 | 0.0172 | 0.004 | >1 |
| 8 | 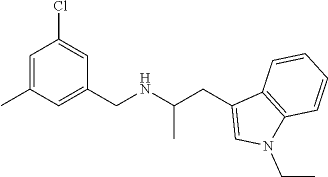 [SMILES: CCn1cc(CC(C)NCc2cc(C)cc(Cl)c2)c2ccccc21] | Not Tested/86.4 | Not Tested | Not Tested | Not Tested |
| 9 | 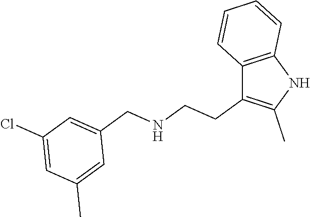 [SMILES: Cc1cc(Cl)cc(CNCCc2c(C)[nH]c3ccccc23)c1] | 0.0103/88.9 | 0.015 | >1 | >1 |
| 10 | 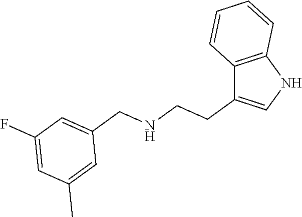 [SMILES: Cc1cc(F)cc(CNCCc2c[nH]c3ccccc23)c1] | Not Tested/72.6 | Not Tested | Not Tested | Not Tested |
| 11 | 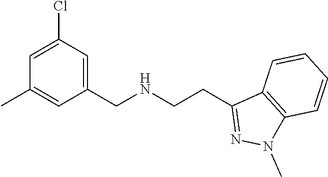 [SMILES: Cc1cc(Cl)cc(CNCCc2nn(C)c3ccccc23)c1] | Not Tested/79.1 | Not Tested | Not Tested | Not Tested |
| 12 | 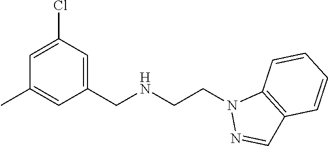 [SMILES: Cc1cc(Cl)cc(CNCCn2ncc3ccccc32)c1] | Not Tested/69.8 | Not Tested | Not Tested | Not Tested |
| 13 | 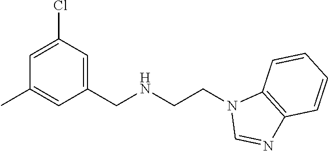 [SMILES: Cc1cc(Cl)cc(CNCCn2cnc3ccccc32)c1] | Not Tested/43.5 | Not Tested | Not Tested | Not Tested |
| 14 | 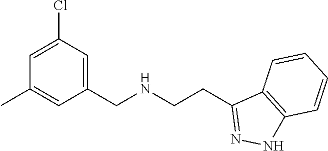 [SMILES: Cc1cc(Cl)cc(CNCCc2n[nH]c3ccccc23)c1] | Not Tested/ | Not Tested | Not Tested | Not Tested |
| 15 | 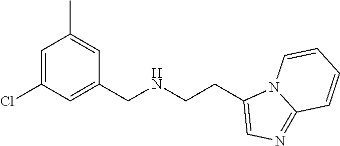 [SMILES: Cc1cc(Cl)cc(CNCCc2cnc3ccccn23)c1] | Not Tested/ | Not Tested | Not Tested | Not Tested |
| 16 | 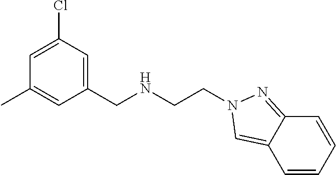 [SMILES: Cc1cc(Cl)cc(CNCCn2cc3ccccc3n2)c1] | Not Tested/ Not Tested | Not Tested | Not Tested | Not Tested |
| 17 | 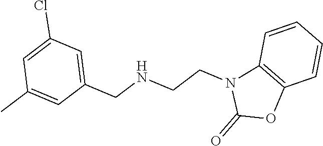 [SMILES: Cc1cc(Cl)cc(CNCCn2c(=O)oc3ccccc32)c1] | Not Tested/−5.4 | Not Tested | Not Tested | Not Tested |
| 18 | 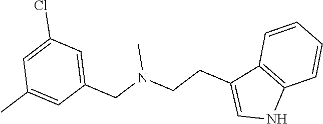 [SMILES: Cc1cc(Cl)cc(CN(C)CCc2c[nH]c3ccccc23)c1] | Not Tested/93.3 | Not Tested | Not Tested | Not Tested |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

We claim:

1. A compound of Formula (I): 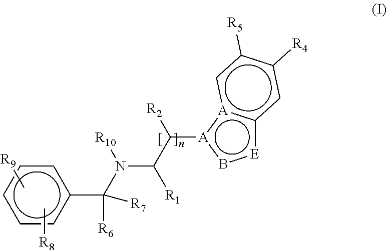
or a pharmaceutically acceptable salt thereof,
wherein,
R₁, R₂, R₆, R₇, and independently are hydrogen, deuterium, alkyl, deuterated alkyl, heteroalkyl, haloalkyl, cycloalkyl, or heterocyclyl;
R₄, R₅, R₈, and R₉ independently are hydrogen, deuterium, alkyl, heteroalkyl, haloalkyl, cycloalkyl, CO₂R₁₁, CONHR₁₁, CO₂N(R₁₁)(R₁₂), CN, or halogen,
wherein R₁₁ and R₁₂ independently are hydrogen, deuterium, heteroalkyl, haloalkyl, cycloalkyl, or heterocyclyl;
R₁₀ is hydrogen, deuterium, or alkyl;
A is C or N;
B is CR₁₃, N, or CO,
wherein R₁₃ is hydrogen, deuterium, alkyl, heteroalkyl, haloalkyl, cycloalkyl, or heterocyclyl;
E is CR₃, NR₃, N, O, or S;
wherein R₃ is hydrogen, deuterium, alkyl, deuterated alkyl, heteroalkyl, haloalkyl, cycloalkyl, or heterocyclyl; and
n is 0 or 1.

2. The compound of claim 1, wherein the compound of Formula (I) is a compound of Formula (I-A): 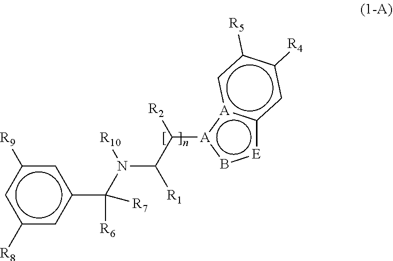
or a pharmaceutically acceptable salt thereof,
wherein,
R₁, R₂, R₆, R₇, and independently are hydrogen, deuterium, alkyl, deuterated alkyl, heteroalkyl, haloalkyl, cycloalkyl, or heterocyclyl;
R₄, R₅, R₈, and R₉ independently are hydrogen, deuterium, alkyl, heteroalkyl, haloalkyl, cycloalkyl, CO₂R₁₁, CONHR₁₁, CO₂N(R₁₁)(R₁₂), CN, or halogen,
wherein R₁₁ and R₁₂ independently are hydrogen, deuterium, heteroalkyl, haloalkyl, cycloalkyl, or heterocyclyl;
wherein when one A is N, R₈ and R₉ are not both hydrogen;
R₁₀ is hydrogen, deuterium, or alkyl;
A is C or N;
B is CR₁₃, N, or CO,
wherein R₁₃ is hydrogen, deuterium, alkyl, heteroalkyl, haloalkyl, cycloalkyl, or heterocyclyl;
E is CR₃, NR₃, N, O, or S;
wherein R₃ is hydrogen, deuterium, alkyl, deuterated alkyl, heteroalkyl, haloalkyl, cycloalkyl, or heterocyclyl; and
n is 0 or 1.

3. The compound of any of claims 1-2, wherein
R₁, R₂, R₃, R₆, and R₇ independently are C₁-C₆ alkyl, C₁-C₆ heteroalkyl, C₁-C₆ haloalkyl, C₃-C₇ cycloalkyl, or C₃-C₇ heterocyclyl;
R₄, R₅, R₈, and R₉ independently are C₁-C₆ alkyl, C₁-C₆ heteroalkyl, C₁-C₆ haloalkyl, C₃-C₇ cycloalkyl, CO₂R₁₁, CONHR₁₁, or CO₂N(R₁₁)(R₁₂);
wherein R₁₁ and R₁₂ independently are C₁-C₆ heteroalkyl, C₁-C₆ haloalkyl, C₃-C₇ cycloalkyl, or C₃-C₇ heterocyclyl;
R₁₀ is C₁-C₆ alkyl;
B is CR₁₃;
wherein R₁₃ is C₁-C₆ alkyl, C₁-C₆ heteroalkyl, C₁-C₆ haloalkyl C₃-C₇, cycloalkyl, or C₃-C₇ heterocyclyl.

4. The compound of any of claims 1-3, wherein n is 1.

5. The compound of any of claims 1-4, wherein R₁ is hydrogen.

6. The compound of any of claims 1-4, wherein R₁ is C₁-C₆ alkyl.

7. The compound of claim 6, wherein R₁ is methyl.

8. The compound of any of claims 1-7, wherein B is CR₁₃.

9. The compound of claim 8, wherein R₁₃ is C₁-C₆ alkyl.

10. The compound of claim 8, wherein R₁₃ is methyl.

12. The compound of claim 8, wherein R₁₃ is hydrogen.

12. The compound of any of claims 1-7, wherein B is CO.

13. The compound of any of claims 1-7, wherein B is N.

14. The compound of any of claims 1-13, wherein one A is N.

15. The compound of any of claims 1-13, wherein one A is N and E is N.

16. The compound of any of claims 1-13, wherein one A is N and E is O.

17. The compound of any of claims 1-15, wherein E is CR₃ or NR₃, wherein R₃ is hydrogen.

18. The compound of any of claims 1-15, wherein E is CR₃ or NR₃, wherein R₃ is C₁-C₆ alkyl.

19. The compound of claim 18, wherein R₃ is methyl.

20. The compound of claim 18, wherein R₃ is ethyl.

21. The compound of any of claims 1-20, wherein R₄ and R₅ are hydrogen.

22. The compound of any of claims 1-20, wherein R₄ is halogen.

23. The compound of claim 22, wherein R₄ is I.

24. The compound of any of claims 1-23, wherein R₅ is C₁-C₆ heteroalkyl.

25. The compound of claim 24, wherein R₅ is methoxy.

26. The compound of any of claims 1-25, wherein R₆ and R₇ are hydrogen.

27. The compound of any of claims 1-26, wherein R₁ is hydrogen.

28. The compound of any of claims 1-26, wherein R₁ is C₁-C₆ alkyl.

29. The compound of claim 28, wherein R₁ is methyl.

30. The compound of any of claims 1-29, wherein R₈ is C₁-C₆ alkyl.

31. The compound of claim 30, wherein R₈ is methyl.

32. The compound of any of claims 1-31, wherein R₉ is halogen.

33. The compound of claim 32, wherein R₉ is Cl.

34. The compound of claim 32, wherein R₉ is F.

35. The compound of any of claims 1-34, wherein R₁₀ is C₁-C₆ alkyl.

36. The compound of claim 35, wherein R₁₀ is methyl.

37. The compound of any of claims 1-36, wherein A and A are not simultaneously N.

38. A compound, or derivative thereof, of Formula (II): 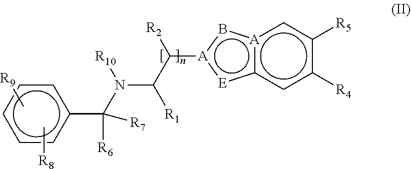
or a pharmaceutically acceptable salt thereof,
wherein,
R₁, R₂, R₆, R₇, and independently are hydrogen, deuterium, alkyl, deuterated alkyl, heteroalkyl, haloalkyl, cycloalkyl, or heterocyclyl;
R₄, R₅, R₈, and R₉ independently are hydrogen, deuterium, alkyl, heteroalkyl, haloalkyl, cycloalkyl, CO₂R₁₁, CONHR₁₁, CO₂N(R₁₁)(R₁₂), CN, or halogen,
wherein R₁₁ and R₁₂ independently are hydrogen, deuterium, heteroalkyl, haloalkyl, cycloalkyl, or heterocyclyl;
R₁₀ is hydrogen, deuterium, or alkyl;
A is C or N;
B is CR₁₃, N, or CO,
wherein R₁₃ is hydrogen, deuterium, alkyl, heteroalkyl, haloalkyl, cycloalkyl, or heterocyclyl;
E is CR₃, NR₃, N, O, or S;
wherein R₃ is hydrogen, deuterium, alkyl, deuterated alkyl, heteroalkyl, haloalkyl, cycloalkyl, or heterocyclyl; and
n is 0 or 1.

39. The compound of claim 38, wherein
R₁, R₂, R₃, R₆, and R₇ independently are C₁-C₆ alkyl, C₁-C₆ heteroalkyl, C₁-C₆ haloalkyl, C₃-C₇ cycloalkyl, or C₃-C₇ heterocyclyl;
R₄, R₅, R₈, and R₉ independently are C₁-C₆ alkyl, C₁-C₆ heteroalkyl, C₁-C₆ haloalkyl, C₃-C₇ cycloalkyl, CO₂R₁₁, CONHR₁₁, or CO₂N(R₁₁)(R₁₂);
wherein R₁₁ and R₁₂ independently are C₁-C₆ heteroalkyl, C₁-C₆ haloalkyl, C₃-C₇ cycloalkyl, or C₃-C₇ heterocyclyl;
R₁₀ is C₁-C₆ alkyl;
B is CR₁₃;
wherein R₁₃ is C₁-C₆ alkyl, C₁-C₆ heteroalkyl, C₁-C₆ haloalkyl C₃-C₇, cycloalkyl, or C₃-C₇ heterocyclyl.

40. The compound of any of claims 38-39, wherein n is 1.

41. The compound of any of claims 38-40, wherein R₁ is hydrogen.

42. The compound of any of claims 38-41, wherein B is N.

43. The compound of any of claims 38-42, wherein one A is N.

44. The compound of any of claims 38-43, wherein E is CR₃ or NR₃, R₃ is hydrogen.

45. The compound of any of claims 38-44, wherein R₄ and R₅ are hydrogen.

46. The compound of any of claims 38-45, wherein R₆ and R₇ are hydrogen.

47. The compound of any of claims 38-46, wherein R₈ is C₁-C₆ alkyl.

48. The compound of claim 47, wherein R₈ is methyl.

49. The compound of any of claims 38-48, wherein R₉ is halogen.

50. The compound of claim 49, wherein R₉ is Cl.

51. The compound of any of claims 38-50, wherein R₂ is hydrogen.

52. The compound of any of claims 38-51, wherein R₁₀ is hydrogen.

53. The compound of any of claims 38-52, wherein A and A are not simultaneously N.

54. A compound selected from:

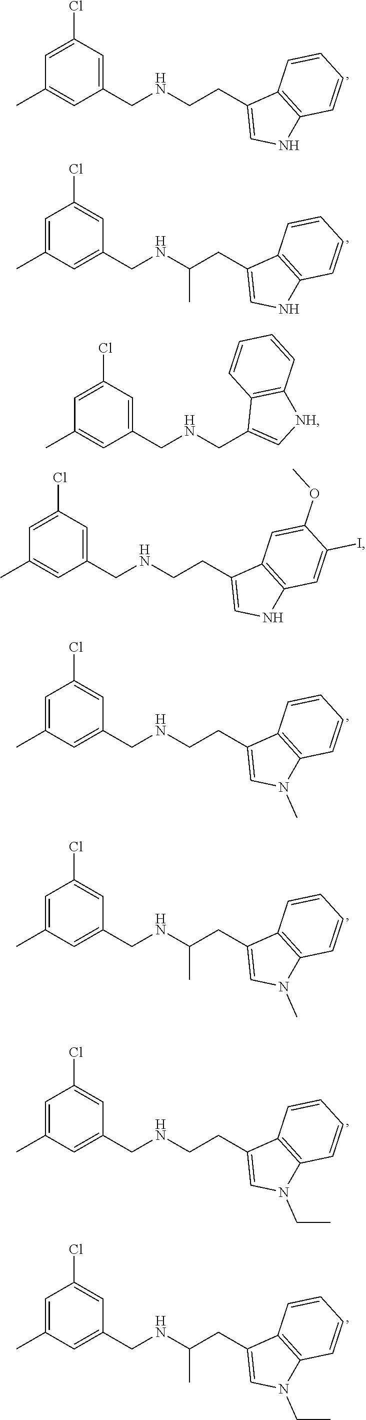

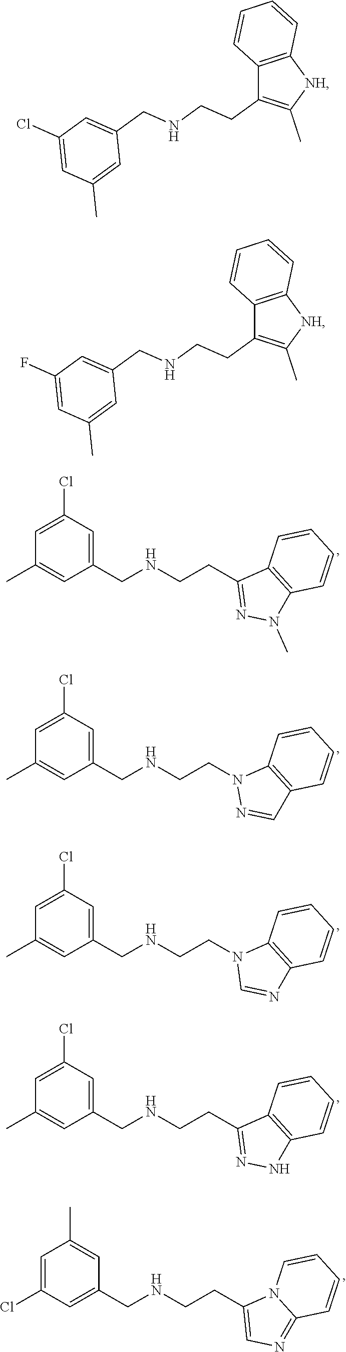

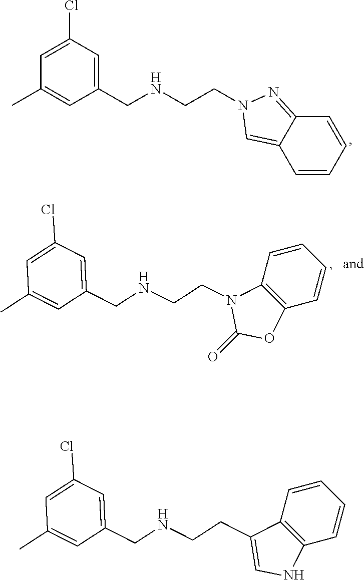 [SMILES: Cc1cc(Cl)cc(CNCCn2cc3ccccc3n2)c1 ; Cc1cc(Cl)cc(CNCCn2c(=O)oc3ccccc32)c1 ; Cc1cc(Cl)cc(CNCCc2c[nH]c3ccccc23)c1]

55. A composition comprising the compound of any of claims 1-54 and a pharmaceutically acceptable carrier.

56. A method for treating one or more conditions that are responsive to serotonin receptor activation, comprising administering to a subject in need thereof an effective amount of the compound of any of claims 1-54 or the composition of claim 55.

57. The method of claim 56, wherein the neurological disorder is major depressive disorder (MDD), treatment resistant depression (TRD), substance use disorder (SUD), an anxiety disorder including acute stress disorder agoraphobia, generalized anxiety disorder, obsessive-compulsive disorder, panic disorder, posttraumatic stress disorder, separation anxiety disorder, social phobia, or specific phobia; an eating disorder including anorexia nervosa or bulimia nervosa; or rumination.

58. A method of activating a 5-HT receptor in a subject comprising administering to the subject a compound of claim 1 or claim 53.

59. The method of claim 58, wherein said 5-HT receptor is a 5-HT2A, 5-HT2B, or 5-HT2C receptor.

60. The method of claim 59, wherein said compound is one of the compounds of claim 52.

61. The compound of claim 54, wherein said compound is

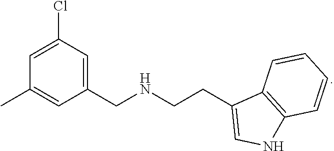 [SMILES: Cc1cc(Cl)cc(CNCCc2c[nH]c3ccccc23)c1]

62. The compound of claim 54, wherein said compound is

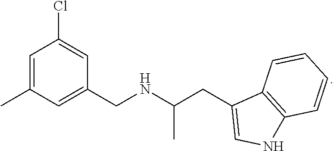 [SMILES: Cc1cc(Cl)cc(CNC(C)Cc2c[nH]c3ccccc23)c1]

63. The compound of claim 54, wherein said compound is

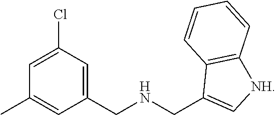 [SMILES: Cc1cc(Cl)cc(CNCc2c[nH]c3ccccc23)c1]

64. The compound of claim 54, wherein said compound is

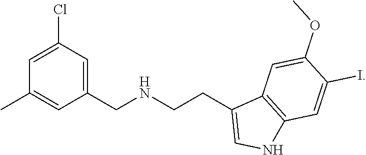 [SMILES: COc1cc2c(CCNCc3cc(C)cc(Cl)c3)c[nH]c2cc1I]

65. The compound of claim 54, wherein said compound is

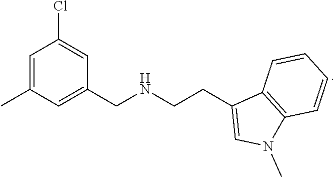 [SMILES: Cc1cc(Cl)cc(CNCCc2cn(C)c3ccccc23)c1]

66. The compound of claim 54, wherein said compound is

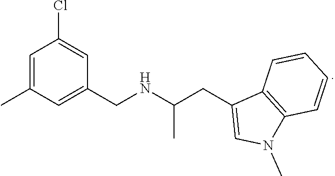 [SMILES: Cc1cc(Cl)cc(CNC(C)Cc2cn(C)c3ccccc23)c1]

67. The compound of claim 54, wherein said compound is

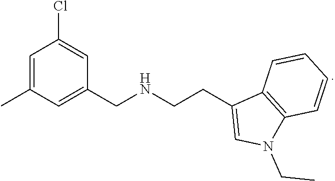 [SMILES: CCn1cc(CCNCc2cc(C)cc(Cl)c2)c2ccccc21]

68. The compound of claim 54, wherein said compound is

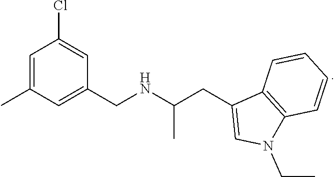 [SMILES: CCn1cc(CC(C)NCc2cc(C)cc(Cl)c2)c2ccccc21]

69. The compound of claim 54, wherein said compound is

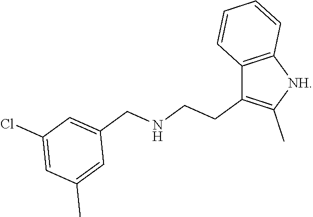 [SMILES: Cc1cc(Cl)cc(CNCCc2c(C)[nH]c3ccccc23)c1]

70. The compound of claim 54, wherein said compound is

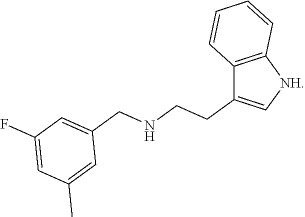 [SMILES: Cc1cc(F)cc(CNCCc2c[nH]c3ccccc23)c1]

71. The compound of claim 54, wherein said compound is

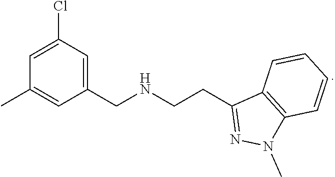 [SMILES: Cc1cc(Cl)cc(CNCCc2nn(C)c3ccccc23)c1]

72. The compound of claim 54, wherein said compound is

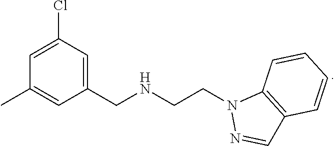 [SMILES: Cc1cc(Cl)cc(CNCCn2ncc3ccccc32)c1]

73. The compound of claim 54, wherein said compound is

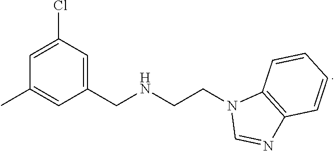 [SMILES: Cc1cc(Cl)cc(CNCCn2cnc3ccccc32)c1]

74. The compound of claim 54, wherein said compound is

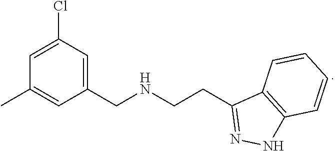 [SMILES: Cc1cc(Cl)cc(CNCCc2n[nH]c3ccccc23)c1]

75. The compound of claim 54, wherein said compound is

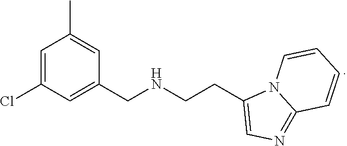 [SMILES: Cc1cc(Cl)cc(CNCCc2cnc3ccccn23)c1]

76. The compound of claim 54, wherein said compound is

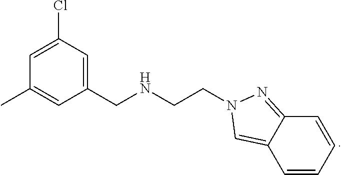 [SMILES: Cc1cc(Cl)cc(CNCCn2cc3ccccc3n2)c1]

77. The compound of claim 54, wherein said compound is

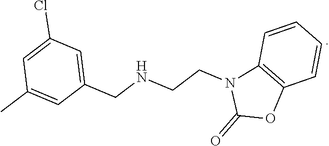 [SMILES: Cc1cc(Cl)cc(CNCCn2c(=O)oc3ccccc32)c1]

78. The compound of claim 54, wherein said compound is 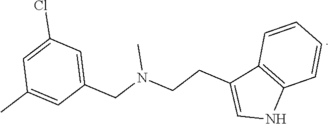 [SMILES: Cc1cc(Cl)cc(CN(C)CCc2c[nH]c3ccccc23)c1]
